(19) United States
(12) Patent Application Publication
(10) Pub. No.: US 2012/0058082 A1
(43) Pub. Date: Mar. 8, 2012

(54) METHODS AND COMPOSITIONS FOR TREATMENT

(75) Inventors: Johanne M. Kaplan, Sherborn, MA (US); Bruce L. Roberts, Southborough, MA (US); William M. Siders, Franklin, MA (US)

(73) Assignee: GENZYME CORPORATION, Cambridge, MA (US)

(21) Appl. No.: 13/319,991

(22) PCT Filed: May 13, 2010
(86) PCT No.: PCT/US10/34780

§ 371 Date: Nov. 10, 2011

Related U.S. Application Data
(60) Provisional application No. 61/177,922, filed on May 13, 2009.

(51) Int. Cl. A61K 38/21; C07K 16/18; A61P 29/00; A61K 39/395; A61P 35/00

(52) U.S. Cl. 424/85.5; 424/172.1; 424/133.1; 530/391.7

(57) ABSTRACT

The invention provides methods for improving the efficacy and reducing side effects of anti-CD52 antibody treatment. The methods can be used to treat patients who are in need of immunoregulation such as lymphocyte depletion and patients who have cancer. Also included are compositions useful for these methods.

This application claims priority from U.S. Provisional Application 61/177,922, filed May 13, 2009. The disclosure of that application is incorporated herein by reference in its entirety.

### FIELD OF THE INVENTION

This invention relates to methods and compositions for treating conditions of the immune system with anti-CD52 antibodies.

### BACKGROUND OF THE INVENTION

CD52 is a cell surface protein expressed at high levels by both normal and malignant B and T lymphocytes (Hale et al., J Biol regal Homeost Agents 15:386-391 (2001); Huh et al., Blood 92: Abstract 4199 (1998); Elsner et at., Blood 88:4684-4693 (1996); Gilleece et al., Blood 82:807-812 (1993); Rodig et al., Clin Cancer Res 12:7174-7179 (2006); Ginaldi et al., Leak Res 22:185-191 (1998)). CD52 is expressed at lower levels by monocytes, macrophages, and eosinophils, with little expression found on mature natural killer (NK) cells, neutrophils, and hematological stem cells. Id. CD52 is also produced by epithelial cells in the epididymis and duct deferens, and is acquired by sperm during passage through the genital tract (Hale et at,, 2001, supra; Domagala et al., Med Sci Monit 7:325-331 (2001)). The exact biological function of CD52 remains unclear but some evidence suggests that it may be involved in T cell migration and co-stimulation(Rowan et al., Int Immunol 7:69-77 (1995); Masuyama et al., J Exp Med 189:979-989 (1999); Watanabe et al., Clin Immunol 120:247-259 (2006)).

Alemtuzumab (CAMPATH-1H®) is a recombinant humanized IgG1 monoclonal antibody directed against human CD52 (hCD52), a 12 amino acid, 28 kD glycosylated glycosylphophatidylinositol (GPI)-linked cell surface protein (Hale et al., Tissue Antigens 35:118-27 (1990); Hale et at, 2001, supra). Alemtuzumab is currently approved as a first line treatment against B-cell chronic lymphocytic leukemia. Treatment with the antibody results in the depletion of CD52+ tumor cells but the mechanism(s) involved are not well-defined. In vitro studies indicate that alemtuzumab is capable of complement-dependent cytotoxicity (CDC), antibody-dependent cell-mediated cytotoxicity (ADCC) as well as induction of apoptosis, but the extent of the role played by these various mechanisms in vivo remains to be established (Golay et al., Haematologica 89: 1476-4483 (2004); Zent et at, Leuk Res 32:1849-1856 (2008); Cruz et al., Leuk Lymphoma 48:2424-2436 (2007); Rowan et al., Immunology 95:427-436 (1998); Smolewski et al., Leuk Lymphoma 46:87-100 (2005); Mone et al., Leukemia 20:272-279 (2006); Nuckel et al, Eur J Pharmacol 14:217-224 (2005)).

Alemtuzumab has also been tested clinically in the context of autoimmune diseases including rheumatoid arthritis, vasculitis, and most notably, multiple sclerosis (MS) (Reiff Hematology 10: 79-93 (2005); Brett et al., Immunology 88:13-19 (1996); Coles et al., J Neurol 253:98-108 (2006); Cox et al., Eur J Immunol 35:3332-3342 (2005); Coles et at, N. Engl J Med 359:1786-1801 (2008)). Recently published results from a Phase II clinical trial in previously untreated relapsing-remitting MS patients showed a 74% reduction in the rate of relapse in patients receiving annual courses of alemtuzumab treatment compared to interferon β-1a given three times per week (Coles et al., 2008, supra). In addition, patients treated with alemtuzumab showed a 71% reduction in the risk for sustained accumulation of disability compared to interferon β-1a-treated patients over a 36 month period. Id. Significant lymphocyte depletion was observed in alemtuzumab-treated patients.

Although the properties of alemtuzumab have been studied in vitro using human peripheral blood lymphocytes, more detailed in vivo mechanism of action studies have been hampered by the fact that the antibody does not cross-react with murine CD52. Homologs of CD52 have been identified in the mouse and several other species that possess very similar signal peptides and 5′ and 3′ untranslated sequences, but the mature peptides are very different amongst species, thus explaining the lack of cross-reactivity (Hale et al., 2001, supra).

### SUMMARY OF THE INVENTION

We have invented new and useful methods and compositions for improving the efficacy and reducing side effects of therapy with anti-CD52 antibodies (e.g., alemtuzumab). Included in the invention are methods for treating a patient in need thereof, comprising: administering to the patient an agent that stimulates neutrophils, or natural killer (NK) cells, or both; and administering to the patient a therapeutically effective amount of an anti-CD52 antibody.

Also included are methods for increasing the efficacy of treatment with an anti-CD52 antibody, comprising: administering to a patient who receives said antibody treatment (e.g., is to undergo or is undergoing, or have undergone) said treatment an agent that stimulates neutrophils, or NK cells, or both.

The invention provides methods of reducing a side effect e.g., infusion reaction, secondary autoimmunity, or development of an antibody response against the administered anti-CD52 antibody) in a patient who receives said treatment with an anti-CD52 antibody, comprising administering to the patient an agent that stimulates neutrophils, NK cells, or both, thereby reducing the effective amount of anti-CD52 antibody needed in the therapy and reducing associated side effects.

The invention provides methods for increasing lymphocyte depletion in a patient who receives treatment with an anti-CD52 antibody, comprising administering to the patient an agent that stimulates neutrophils, or NK cells, or both. In some embodiments, the patient has an abnormally low neutrophil count (e.g., neutropenia) prior to the antibody treatment or as a result of the antibody treatment.

The invention also provides methods for increasing CD4+H-CD25+FoxP3+ regulatory T (Treg) cells in a patient who receives anti-CD52 antibody therapy, comprising administering to the patient an agent that stimulates the regulatory neutrophils, or NK cells, or both. In some embodiments, the methods further comprise administering to the patient an agent that stimulates neutrophils, or NK cells, or both. Treg stimulators include, including, without limitation rapamycin, a TGF-β (active or latent TGF-β1, TGF-β2, TGF-β3, TGF-β4, and TGF-β5), IL-10, IL-4, IFN-α, vitamin D3, dexamethasone, and mycophenolate mofetil.

In the methods of this invention, the agent for stimulating neutrophils and/or NK cells may be, for example, granulocyte monocyte colony stimulating factor (GM:CSF) (e.g., sargramostim), granulocyte colony stimulating factor (G-CSF), interferon-gamma (IFN-β, e.g., ACTIMMUNE®), a CXC chemokine receptor 4 antagonist (e.g., plerixafor), or a CXC chemokine receptor 2 agonist, In the methods of this invention, the administering steps may be concurrent or sequential. For example, the Treg stimulator or the neutrophil/NK stimulators can be administered before, during, or after the anti-CD52 antibody therapy.

The methods of this invention can be used on patients who suffer from inflammatory conditions, autoimmune diseases, and cancer. For example, the patient that can be treated with the methods of this invention may suffer multiple sclerosis, rheumatoid arthritis (RA), vasculitis, myositis, scleroderma, aplastic anemia, or systemic lupus erythematosus (or lupus). Or the patients may suffer malignancy of CD52-expressing cells (e.g., T cell malignancy or B malignancy), including, e.g., leukemia, lymphoma, low grade/follicular non-Hodgkin's lymphoma (NHL), small lymphocytic (SL) NHL, intermediate grade/follicular NHL, intermediate grade diffuse NHL, chronic lymphocytic leukemia (CLL), high grade immunoblastic NHL, high grade lymphoblastic NHL, high grade small noncleaved NHL, bulky disease NHL, mantle cell lymphoma, AIDS-related lymphoma and Waldenstrom's Macroglobulinemia. In some embodiments, the patient is in need of, is undergoing, or having undergone, a transplantation (e.g., a stem cell transplant, an infusion of autologous of allogeneic T or a solid organ transplant), and the methods of this invention can be used, for example, to prevent or alleviate GVHD. In some embodiments, the patient has neovascularization and the anti-CD52 antibody therapy is used to treat the neovascularization (e.g., tumor angiogenesis), Cancers treatable by methods of this invention includes: breast cancer, lung cancer, ovarian cancer, glioma, colorectal cancer, etc.

The invention also provides compositions and kits for use in the methods. For example, the invention provides kits comprising (a) an anti-CD52 antibody; and (b) an agent that stimulates neutrophils or NK or Treg cells. Also by way of example, the invention provides immunoconjugates comprising an anti-CD52 antibody fused (via genetic modifications or chemical conjugation) to an agent that stimulates neutrophils or NK or Treg cells, and pharmaceutical compositions comprising such an immunoconjugate and a pharmaceutically acceptable carrier.

### BRIEF DESCRIPTION OF THE FIGURES

FIG. 1 is a graph showing levels of CD52 expression on immune cell populations. Human CD52 expression was quantified on the indicated cell populations from the spleen, bone marrow (BM) and thymus of hCD52 transgenic mice. Using multi-parameter flow cytometry, hCD52 mean fluorescence intensities were quantified and used to calculate the number of hCD52 molecules/cell. The cell populations examined included B220⁺ B cells, CD4⁺ T cells, CD4⁺CD25⁺FoxP3⁻ T cells (CD4 Treg), CD8⁺ T Cells, CD11 b⁺CD11c⁻ macrophages, Gr-1⁺ neutrophils, NK1.1⁺CD49b⁺ mature NK cells, c-Kit⁺Sca⁺CD45⁻ bone marrow stem cells, CD4 and CD8 single positive thymocytes, double positive and double negative thymocytes. Non-transgenic (NTG) B220⁺ B cells are shown as a representative population to demonstrate the level of background staining for all NTG cell populations. Error bars indicate the standard error of the mean (SEM) of 6 animals/group.

FIGS. 2A-2B depict immune status of hCD52 transgenic mice, Wild type (WT) CD-1 mice and hCD52 transgenic (Tg) CD-1 mice were immunized with a non-replicating adenovirus (Ad) vector, Three weeks later, serum samples and spleens were collected from individual mice to assess humoral and cellular responses to Ad. Mean Ad-specific antibody titers ±SEM of serum samples from individual naëve or immunized mice (n=3) were plotted in FIG. 2A; Mean Ad-induced proliferation ±SEM of spleen cells from individual naëve or immunized mice (n=3) were plotted in FIG. 2B. There were no significant differences between the responses of wild type and hCD52 transgenic mice (p>0.05).

FIGS. 3A-3F show immune cell depletion after treatment with alemtuzumab. Absolute numbers of immune cell populations remaining at 72 hours after the administration of various intravenous (i.v.) doses of alemtuzumab were assessed. Results shown are the mean ±SEM of individual mice (n=5) and are expressed as the percent of cells remaining after treatment relative to the number of cells present in vehicle-treated control mice (% Control). The organs examined included the blood (FIG. 3A), spleen (FIG. 3B), inguinal lymph nodes (FIG. 3C) and thymus (FIG. 3D). The cell populations analyzed consisted of CD4⁺ T cells, CD8⁺ T cells, single positive (SP) and double positive (DP) thymocytes, B220⁺ B cells, NK1.1⁻CD49b⁺ NK cells and Gr-1⁺ neutrophils. Analysis of remaining numbers of CD4⁺CD25⁺FoxP3⁺ T cells (CD4 Treg), compared to total CD4⁺ T cells was also performed for the blood (FIG. 3E) and spleen (FIG. 3F).

FIG. 4 depicts the pattern of lymphocyte repopulation after treatment with alemtuzumab. Blood samples were collected at various time points following the intraperitoneal (i.p.) administration of 10 mg/kg alemtuzumab and the absolute numbers of CD4⁺ T cells, CD8⁺ cells and CD19⁺ B cells were assessed. Results shown are the mean ±SEM of individual mice (n=8) and are expressed as the percent of cells remaining after treatment relative to the number of cells present in untreated, age-matched control mice (% Control).

FIGS. 5A-5B show results of studies on mechanism of lymphocyte depletion by alemtuzumab. Immune effector arms were selectively inactivated to study the impact on the lymphocyte-depleting activity of alemtuzumab. Mice were either left untreated (intact) or were treated with cobra venom factor to remove complement (C′ removed), anti-asialo-GM1 to remove NK cells (NK removed) or anti-Gr-1 to remove neutrophils (PMN removed) prior to the administration of alemtuzumab (0.1 mg/kg, i.p,). Absolute numbers of CD4+ T CD8+ T cells and CD19⁺ B cells remaining in the blood (FIG. 5A) and spleen (FIG. 5B) at 72 hours post-alemtuzumab were assessed. Results shown are the mean ±SEM of individual mice (n=7) and are expressed as the percent of cells remaining after treatment relative to the number of cells present in untreated, control mice (% Control). (*p<0.05, **p<0.01 vs intact mice)

FIGS. 6A-6C depict results of induction of serum cytokines by alemtuzumab. Mice were injected with various doses of alemtuzumab (0.5, 1, or 5 mg/kg, i.p.) or with PBS or Remicade® as an isotype control (Ctl Ig, 5 mg/kg). Remicade® is a human IgG1 specific for human INF-α and does not cross-react with murine TNF-α. Serum samples were collected at 1 hour, 2 hours, 4 hours, 24 hours post-dosing with alemtuzumab and cytokine levels were measured with a multiplex mouse cytokine assay kit. Data are shown for the 2-hour cytokine peak only and represent the mean ±SEM of individual mice n=5) for TNF-α (FIG. 6A), IL-6 (FIG. 6B), and MCP-1 (FIG. 6C). (*p<0.01 vs PBS)

FIGS. 7A-7B show results of studies on mechanism of cytokine induction by alemtuzumab. Immune effector arms were selectively inactivated to study the impact on the cytokine-inducing activity of alemtuzumab. Serum levels of INF-α (FIG. 7A) and MCP-1 (FIG. 7B) at 2 hours post-alemtuzumab (0.1 mg/kg, i.p.) are shown for mice that were either left untreated (Ab) or were treated with cobra venom factor to remove complement (Ab minus C′), anti-asialo-GM1 to remove NK cells (Ab minus NK) or anti-Gr-1 to remove neutrophils (Ab minus PMN) prior to the administration of alemtuzumab. Results shown are the mean ±SEM of individual mice (n=5). Background (baseline) levels of serum cytokines in untreated mice are also shown. (*p<0.01, **p<0.05 vs baseline)

FIGS. 8A-8D show activity of alemtuzumab in disseminated tumor models. Groups of 10 mice were injected with tumor cells and treatment with alemtuzumab (Alem) was initiated 1 to 14 days post-tumor cell injection (10 mg/kg i.p., twice weekly). Animals were sacrificed when hind limb paralysis resulting from central nervous system involvement was first observed. Results shown represent the percent survival of each treatment group over time in the B104 (FIG. 8A), Raji (FIG. 8B), Ramos (FIG. 8C), and IM-9 (FIG. 8D) xenograft tumor models. MS=median survival, (^{a}p<0.0001, ^{b}p=0.0002, ^{c}p>0.2 vs vehicle control group.)

FIGS. 9A-9D show activity of alemtuzumab in subcutaneous tumor models. Groups of 10 mice were injected subcutaneously (s.c.) with tumor cells and treatment with alemtuzumab (Alem) was initiated 1 to 14 days post-tumor cell injection (10 mg/kg i.p., twice weekly). Tumor size was measured twice per week in the B104 (FIG. 9A), MC/CAR. (FIG. 9B), Raji (FIG. 9C), and CHO-CD52 (CHO cells stably transduced with hCD52) (FIG. 9D) xenograft tumor models. Animals were sacrificed when tumor size reached ≧1500 mm³. Mean tumor size ±SEM is shown for each time point.

FIGS. 10A-10D show that inactivation of immune effector mechanisms inhibits the anti-tumor activity of alemtuzumab. FIG. 10A shows the antibody-dependent cell-mediated cytotoxicity (ADCC) activity of deglycosylated (circle) and unmodified (square) alemtuzumab (Alem) against CD52 positive CHO-CD52 cells. Purified human IgG was used as a negative control (diamond). Human peripheral blood mononuclear cells (PBMCs) were used as effector cells at an effector:target (E:T) ratio of 50:1 and the antibodies were added at concentrations ranging from 0.5-10 μg/ml. FIG. 10B shows the complement-dependent cytotoxicity (CDC) activity of deglycosylated and unmodified alemtuzumab against CD52 positive CHO-CD52 cells, using purified human IgG as a negative control. Deglycosylation abolished both the ADCC and CDC activity of alemtuzumab. FIG. 10C shows the anti-tumor activity of deglycosytated and unmodified alemtuzumab in the s.c. CHO-CD52 tumor model. Antibody treatment (10 mg/kg, i.p., twice weekly) was initiated 1 day post-tumor cell injection. Unmodified alemtuzumab had significant anti-tumor activity (p=0.0007 vs untreated) while the deglycosylated form failed to have any impact on tumor growth (p=0.2188 vs untreated). FIG. 10D shows that removal of complement and mediators of ADCC removed the ability of alemtuzumab to control tumor growth (p=0.1795 vs untreated) compared to its activity in intact mice (p=0.0001 vs untreated).

FIGS. 11A-11B show involvement of NK cells and neutrophils in the anti-tumor activity of alemtuzumab. The anti-tumor activity of alemtuzumab (10 mg/kg i.p, twice weekly starting 1 day post-tumor cell injection for CHO-CD52 and 11 days post-tumor cell injection for B104) was tested in mice selectively depleted of complement, NK cells or neutrophils. FIG. 11A shows that in the CHO-CD52 s.c. model, removal of complement had no effect on anti-tumor activity (100% survival). Removal of NK cells reduced the activity of alemtuzumab but significant anti-tumor protection was still achieved (p=0.0093 vs vehicle) while removal of neutrophils abolished the activity of the antibody (p=0.8620 vs vehicle). FIG. 11B shows that in the B104 s.c. model, removal of complement or NK cells alone reduced the anti-tumor activity of alemtuzumab which however remained significant (p=0.0010 and p=0.0045 vs untreated, respectively) while removal of neutrophils abolished anti-tumor protection by alemtuzumab (p=0.1022 vs untreated). MS=median survival.

FIGS. 12A-12B show activity of mouse neutrophils. FIG. 12A shows the ADCC activity of mouse neutrophils against various tumor cell lines (E:T ratio 200:1), measured in the presence of 10 μg/ml alemtuzumab or infliximab as a negative control antibody. Mean percent lysis ±SEM values are shown. ADCC activity was observed against all cell lines except for IM-9, which expresses very low levels of CD52. FIG. 12B shows the anti-tumor activity of alemtuzumab (10 mg/kg i.p. twice weekly starting 4 days post-tumor cell injection), tested in the Raji s.c. model in mice that did or did not receive G-CSF (20 μg/mouse twice weekly) to increase the number of effector neutrophils. Treatment with G-CSF significantly increased the anti-tumor activity of alemtuzumab (p=0.0291 vs alemtuzumab alone).

FIGS. 13A-B show the genomic sequence of a human CD52 antigen (NCBI NC_{—}000001.9).

FIG. 13C shows the cDNA sequence of a human CD52 antigen (NCBI CCDS ID CCDS30647.1).

FIGS. 14A-14B show the impact of co-administering Alemtuzumab/CAMPATH®-1H and G-CSF/NEUOPOGEN® on lymphocyte depletion in human CD52 transgenic mouse.

FIGS. 15A-15B show the impact of co-administering Alemtuzumab/CAMPATH®-1H and GM-CSF/LEUKINE® on lymphocyte depletion in human CD52 transgenic mouse.

FIGS. 16A-16F show the kinetics of lymphocyte depletion in human CD52 transgenic mouse in response to co-administration of Alemtuzumab/CAMPATH®-1H and G-CSF/NEUOPOGEN®.

### DETAILED DESCRIPTION OF THE INVENTION

This invention is based on our discovery that neutrophils and natural killer (NK) cells are effector cells involved in the antibody-dependent cell-mediated cytotoxicity (ADCC) of alemtuzumab. We have discovered that stimulation (including activation and recruitment) of neutrophils and/or NK cells has a synergistic effect on anti-CD52 antibody therapies. We have further discovered that stimulation of neutrophils and/or NK cells allows a clinician to reduce the dose and therefore certain side effects of an anti-CD52 antibody therapy without compromising the efficacy of the therapies. We have also discovered that anti-CD52 antibody therapies do not deplete CD4⁺CD25⁺FoxP3⁺ regulatory T cells to the same extent as other CD4⁺T cells, even though both populations express equivalent levels of CD52 on their surface; this discovery allows for additional methods of improving anti-CD2 antibody therapies.

The above discoveries were made possible in part by a hCD52 transgenic mouse model that we created (see Examples below). Due to the low cross-reactivity of anti-hCD52 antibodies with murine CD52, the hCD52 transgenic mouse model allowed for in-depth characterization of the biological impact and mechanism of lymphocyte depletion by alemtuzumab in vivo. The hCD52 transgenic mice effectively reproduced the CD52 tissue distribution and levels observed in humans and responded to treatment with alemtuzumab in a similar manner. Our hCD52 transgenic mice did not display any phenotypic abnormalities and were able to mount normal immune responses. In this transgenic model, we have shown that both lymphocyte depletion and cytokine induction by alemtuzumab are largely independent of complement and are mediated by neutrophils and NK cells, We have shown that removal of neutrophils and natural killer (NK) cells strongly inhibit the activity of alemtuzumab, while removal of complement by treatment with cobra venom factor has no impact.

Our discoveries in the transgenic mouse model are supported by our studies in two additional types of tumor mouse models that we have created (see Examples below). In the disseminated tumor mouse models, we injected intravenously hCD52+ tumor cells into the mice, where the tumor cells seeded in multiple organs, giving rise to tumors. In the subcutaneous tumor mouse models, we injected hCD52+ tumor cells into the flanks of the mice. We treated mice in both types of models with alemtuzumab. We found that the anti-tumor activity of alemtuzumab in vivo in these animals was primarily dependent on ADCC mediated by neutrophils and NK cells, as evidenced by the loss of tumor growth inhibition caused by removal of these cell populations with antibodies to Gr-1 and asialo-GM-1, respectively. Again, in activation of complement by treatment with cobra venom factor had no significant impact on the protective activity of alemtuzumab. We further demonstrated that increasing the number of circulating neutrophils by treatment with G-CSF enhanced the anti-tumor activity of alemtuzumab.

In sum, our findings demonstrate for the first time that anti-CD52 antibodies such as alemtuzumab deplete lymphocytes by neutrophil- and NK-mediated ADCC. Accordingly, our invention provides methods for improving anti-CD52 antibody therapies, including enhancing their therapeutic efficacy and reducing non-ADCC related side effects. These methods can be used on patients who are in need of treatment with an anti-CD52 antibody, including patients who suffer from a lymphocyte hyper-proliferative condition, e.g., T or B cell malignancies including leukemia such as non-Hodgkin's lymphoma or lymphoma such as B cell chronic lymphocytic leukemia; or from an autoimmune disease, e.g., multiple sclerosis, systemic lupus, rheumatoid arthritis, vasculitis, psoriasis, myositis, scleroderma, aplastic anemia, and colitis.

### Anti-CD52 Antibody Therapies

The anti-CD52 antibody therapies encompassed by this invention include any treatment regimens using an anti-CD52 antibody, including antibodies of any suitable isotype (IgM, IgD, or IgE) and subtype, such as IgG1, IgG2, IgG3, or IgG4. Useful antibodies also include those whose constant/Fc regions have been modified and bind to a Fe receptor on neutrophils and/or NK cells with the same or better affinity or otherwise with enhanced effector functions. The anti-CD52 antibodies useful in this invention are those that bind specifically to a CD52, and do not bind specifically to non-CD52 molecules. Specific binding between an anti-CD52 antibody and CD52 can be determined, for example, by measuring EC₅₀ of the antibody's binding to CD52+ cells by flow cytometry. Specific binding may be indicated by an EC₅₀ value of, e.g., 0.5-10 μg/ml. For clinical applications, the anti-CD52 antibodies may preferably be monoclonal, with pharmaceutically acceptable purity. The antibodies may be administered in any suitable method, optionally with a pharmaceutically acceptable carrier, at a therapeutically effective amount, e.g., an amount that can help a patient to reach a desired clinical endpoint.

Examples of anti-CD52 antibodies useful in this invention are humanized or human antibodies against hCD52, for example, alemtuzumab (e.g., CAMPATH-1H®) and variants thereof. An example of a human CD52 antigen polypeptide sequence is:

(SEQ ID NO: 1; NCBI Accession No. NP_001794)
MKRFLFLLLT ISLLVMVQIQ TGLSGQNDTS QTSSPSASSN
ISGGIFLFFV ANAIIHLFCF S

A mature human CD52 antigen is considerably shorter (Xia et al., Eur J Immunol. 21(7):1677-84 (1991)) and is glycosylated. An example of a wildtype mature human CD52 has the following sequence: GQNDTSQTSSPS (SEQ ID NO:2). The antibody preferably binds specifically to human CD52 when the patient to be treated is a human patient. In some embodiments, the antibodies of this invention binds to hCD52 with the sequence of SEQ ID NO:2. In some embodiments, the antibody may bind to allelic variants of this CD52 sequence.

Useful antibodies include, without limitation, those that compete with alemtuzumab for binding to hCD52, and/or bind the same or an overlapping epitope as alemtuzumab. Antibodies that bind to other epitopes on CD52 can also be used. To minimize immunogenicity, it may be preferred to use human, humanized and chimeric anti-CD52 antibodies for the methods of this invention, especially in cases where repeated administration of the antibody is needed. In some embodiments, humanized anti-human CD52 antibodies described in International Application PCT/US2010/034704 can be used. The antibodies useful in the methods of this invention can be of any isotype or sub-isotype with the ADCC effector function. For example, a suitable isotype is IgG, and a suitable subtype can be IgG1, IgG2, IgG3, or IgG-4.

If desired, the anti-CD52 antibodies useful in this invention can comprise a detectable label to allow, e.g., monitoring in therapies, diagnosis, or assays. Suitable detectable labels include, for example, a radioisotope (e.g., as Indium-111, Technnetium-99m or Iodine-131), positron emitting labels (e.g., Fluorine-19), paramagnetic ions (e.g., Gadlinium (III), Manganese (II)), an epitope label (tag), an affinity label (e.g., biotin, avidin), a spin label, an enzyme, a fluorescent group, or a chemiluminescent group. When labels are not employed, complex formation can be determined by surface plasmon resonance, ELISA, flow cytometry, or other suitable methods. Anti-CD52 antibodies used in this invention may be conjugated to another therapeutic agent, such as a bioactive compound (e.g., cytokines, and cytotoxic agents). Anti-CD52 antibodies used in the invention also may be conjugated, via, for example, chemical reactions or genetic modifications, to other moieties (e.g., pegylation moieties) that improve the antibodies pharmacokinetics such as half-life. In some embodiments, the anti-CD52 antibodies used in this invention can be linked to a suitable cytokine (e.g., the neutrophil/NK stimulator described below) via, e.g., chemical conjugation or genetic modifications (e.g., appending the coding sequence of the cytokine in frame to an antibody coding sequence, thereby creating an antibody:cytokine fusion protein).

Neutrophil and/or NK Cell Stimulators

This invention provides methods for increasing the lymphocyte-depleting efficacy of an anti-CD52 antibody by stimulating neutrophils and/or NK cells in a patient. Stimulating neutrophils and/or NK cells include, without limitation, (1) increasing their rates of division, (2) increasing their cell surface expression of the Fc receptors corresponding to the isotype of the anti-CD52 antibody (e.g., FcγRIIIa and FcγRIIIb, FcγRII, FcγRI, and FcαRI), (3) mobilizing and increasing the number of circulating cells, (4) recruiting the cells to target sites (e.g., sites of tumors, inflammation, or tissue damage), (5) and increasing their cytotoxic activity.

Examples of agents that stimulate neutrophils and/or NK cells include, for example, granulocyte monocyte colony stimulating factor (GM-CSF) (e.g., LEUKINE® or sargramostim and molgramostim); granulocyte colony stimulating factor (G-CSF) (e.g., NEUPOGEN® or filgrastim, pegylated filgrastim, and lenograstim,); interferon-gamma (IFN-γ, e.g., ACTIMMUNE®); CXC chemokine receptor 4 (CXCR4) antagonists (e.g., MOZOBIL™ or plerixafor); and CXC chemokine receptor 2 (CXCR2) agonists.

The neutrophil and/or NK stimulator can be administered prior to, during, or after administration of the anti-CD52 antibody, to improve the efficacy of the anti-CD52 antibody. In an anti-CD52 therapeutic regimen involving multiple administrations of the anti-CD52 antibody, the neutrophil and/or NK stimulator can be administered once or more than once at any time point deemed appropriate by a health care provider. During anti-CD52 antibody treatment, the neutrophil count of the patient may be monitored periodically to ensure optimal treatment efficacy. The neutrophil count of the patient also can be measured prior to the start of the anti-CD52 antibody treatment. The stimulator's amount can be adjusted based on the patient's neutrophil count. A higher dose of the stimulator may be used if the patient has a lower than normal neutrophil count. During periods of neutropenia, which may be caused by treatment with the anti-CD52 antibody, a higher dose of the neutrophil stimulator may also be administered to maximize the effect of the anti-CD52 antibody.

Because neutrophil and/or NK stimulation improves the efficacy of an anti-CD52 antibody treatment, one may be able to use less antibody in a patient while maintaining similar treatment efficacy. Using less anti-CD52 antibody while maintaining treatment efficacy may help reduce side effects of the anti-CD52 antibody, which include infusion reactions, immune response in the patient against the administered antibody as well as development of secondary autoimmunity (autoimmunity that arises during or after anti-CD52 antibody treatment).

### Increasing CD4⁺CD25⁺FoxP3⁺ Regulatory T Cells

We have discovered that anti-CD52 antibodies tend to deplete CD4⁺CD25⁺FoxP3⁺ regulatory T cells to a lesser extent as compared to other CD4+ T cells. Regulatory T cells (also known as “Treg” or suppressor T cells) are cells that are capable of inhibiting the proliferation and/or function of other lymphoid cells via contact-dependent or contact-independent (e.g., cytokine production) mechanisms. Several types of regulatory T cells have been described, including γδ T cells, natural kilter T (NKT) cells, CD8⁺T cells, CD4⁺T cells, and double negative CD4⁻CD8 T cells. See, e.g., Bach et al., Immunol. 3:189-98 (2003). CD4³⁰ CD25⁺FoxP3⁺ regulatory T cells have been referred to as “naturally occurring” regulatory T cells; they express CD4, CD25 and forkhead family transcription factor FoxP3 (forkhead box p3).

In some embodiments of this invention, an increase of Tregs may be desired for enhancing the efficacy of the anti-CD52 antibody therapy, e.g., further reducing symptoms of the autoimmune disease being treated. In those embodiments, one can administer an agent that stimulates CD4⁺CD25⁺FoxP3⁺ regulatory T cells. The agent may, for example, activate those T cells, stabilize and/or expand the population of the cells, mobilize and increase circulation of the cells, and/or recruit the cells to target sites. Examples of such agents are rapamycin (e.g., L-rapamycin), active or latent TGF-β (e.g., TGF-β1, TGF-β2, TGF-β3, TGF-β4, and TGF-β5), IL-10, IL-4, IFN-α, vitamin D3, dexamethasone, and mycophenotate mofetil (see, e.g., Barrat. et al., J. Exp. Med 195:603-616 (2002); Gregori et al., J Immunol. 167: 1945-1953 (2001); Battaglia et al., Blood 105: 4743-4748 (2005)).

### Therapeutic Uses

In some embodiments, the methods of this invention can be used on patients who suffer from autoimmune diseases. The patients may be treated when the disease is active (e.g., in relapse), or in remission, as needed. Examples of autoimmune diseases include, but are not limited to, Addison's disease, hemolytic anemia, antiphospholipid syndrome, rheumatoid arthritis, dermatitis, allergic encephalomyelitis, glomerulonephritis, Goodpasture's syndrome, Graves' disease, multiple sclerosis, vasculitis, scleroderma, myasthenia gravis, neuritis, ophthalmia, bullous pemphigoid, pemphigus, polyendocrinopathies, Purpura, Reiter's disease, Stiff-Man syndrome, autoimmune thyroiditis, lupus, autoimmune pulmonary inflammation, Guillain-Barre syndrome, insulin dependent diabetes mellitus, and autoimmune inflammatory eye disease.

In some embodiments, the methods of this invention can be used on patients who suffer from various manifestations of lupus including, without limitation, systemic lupus erythematosus, lupus nephritis, cutaneous lupus erythematosus, CNS lupus, cardiovascular manifestations, pulmonary manifestations, hepatic manifestations, haematological manifestations, gastrointestinal manifestations, museuloskeletal manifestations, neonatal lupus erythematosus, childhood systemic lupus erythematosus, drug-induced lupus erythematosus, anti-phospholipid syndrome, and complement deficiency syndromes resulting in lupus manifestations (see, e.g., Robert G. Lahita, Editor, Systemic Lupus Erythematosus, 4th Ed., Elsevier Academic Press, 2004).

In some embodiments, the methods of this invention can be used to treat various types of multiple sclerosis, including relapsing-remitting, secondary progressive, primary progressive, and progressive relapsing multiple sclerosis ((Lublin et al., Neurology 46 (4), 907-11 (1996).

The methods of this invention also can be used to treat various cancers, including inhibiting angiogenesis in tumors (see, e.g., Pulaski et al., J. Translational Med. 7:49 (2009)), and killing CD52+ cancerous cells. The methods can also be used as part of a conditioning regimen to prepare a patient before a transplantation (e.g., stem cell transplantation, an infusion of autologous or allogeneic T cells, and a solid organ transplantation). The methods can also be used to enrich hematopoietic stem cell population. The methods can also be used to treat neovascularization.

In this invention, an effective amount of anti-CD52 antibody for treating a disease is an amount that helps the treated subject to reach one or more desired clinical end points. For example, for lupus, clinical endpoints can be measured by monitoring of an affected organ system (e.g., hematuria and/or proteinuria for lupus nephritis) and/or using a disease activity index that provides a composite score of disease severity across several organ systems (e.g., BILAG, SLAM, SLEDAI, ECLAM). See, e.g., Mandl et al., “Monitoring patients with systemic lupus erythematosus” in Systemic Lupus Erythematosus, 4^{th} edition, pp, 619-631, R. G. Lahita, Editor, Elsevier Academic Press, (2004).

In another example of autoimmune disease, multiple sclerosis, diagnosis is made by, for example, the history of symptoms and neurological examination with the help of tests such as magnetic resonance imaging (MRI), spinal taps, evoked potential tests, and laboratory analysis of blood samples. In MS, the goal of treatment is to reduce the frequency and severity of relapses, prevent disability arising from disease progression, and promote tissue repair (Compston and Coles, 2008). Thus, an amount of anti-CD52 antibody that helps achieve a clinical endpoint consistent with that goal is an effective amount of antibody for the treatment.

In this invention, the anti-CD52 antibody and an auxiliary agent (e.g., an agent that stimulates neutrophils and/NK (cells or an agent that stimulates Tregs) are administered to a patient in need thereof simultaneously, or sequentially, or both. For example, the antibody and the agent are formulated together into a single dosage form that can release the two components either concurrently or consecutively (e.g., controlled release or sustained release) to the patient. The antibody and the agent can also be formulated apart in separate dosage forms that can be taken by the patient either at substantially the same time or at consecutive times. The two administrations are through either the same route or two different routes. The antibody and the agent, when formulated in separate forms, also can be released either concurrently or consecutively (e.g., controlled release or sustained release) in the patient. In some embodiments, the antibody and the agent are provided as a kit.

### Pharmaceutical Compositions and Kits

This invention also provides compositions comprising an immunoconjugate comprising an anti-CD52 antibody fused to a stimulatory agent (e.g., a Treg, neutrophil, or NK cell stimulator) and a pharmaceutically acceptable carrier.

As used herein, “pharmaceutically acceptable carrier” means any and all solvents, dispersion media, coatings, anti-bacterial and anti-fungal agents, isotonic and absorption delaying agents, and the like that are physiologically compatible. Some examples of pharmaceutically acceptable carriers, merely by way of illustration, are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the antibody.

The compositions may be in a variety of forms, for example, liquid, semi-solid and solid dosage forms, such as liquid solutions (e.g., injectable and infusible solutions), dispersions or suspensions, tablets, pills, powders, liposomes and suppositories. The form depends on the intended mode of administration and therapeutic application. Typical compositions are in the form of injectable or infusible solutions, such as compositions similar to those used for passive immunization of humans. In one embodiment, the composition is administered by intravenous infusion or injection. In still another embodiment, the composition is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The compositions of the invention may be administered by a variety of methods known in the art, although for many therapeutic applications, the preferred route/mode of administration is subcutaneous, intramuscular, or intravenous infusion. As will be appreciated by the skilled artisan, the route and/or mode of administration will vary depending upon the desired results. Other modes of administration include intraperitoneal, intrabronchial, transmucosal, intraspinal, intrasynovial, intraaortic, intranasal, ocular, otic, topical and buccal, and intratumor.

In some embodiments, the active compound of the antibody compositions may be prepared with a carrier that will protect the active ingredient (e.g., the immunoconjugate against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art.

This invention also provides kits comprising an anti-CD52 antibody and an agent that stimulates neutrophils or NK cells or Tregs. A kit may include instructions for use in a therapeutic method, as well as packaging material such as, but not limited to, ice, dry ice, STYROFOAM™, foam, plastic, cellophane, shrink wrap, bubble wrap, cardboard and starch peanuts.

### Transgenic Mouse Model

This invention also provides a transgenic mammal (e.g., mouse) expressing human CD52. The transgenic mouse model generated in this invention can effectively reproduce the CD52 tissue distribution and levels observed in humans and respond to treatment with alemtuzumab in a similar manner. In some embodiments, the transgenic mammal has a heterozygous or homozygous lull mutation in its endogenous CD52 gene. The transgenic mouse model of the invention can be used to investigate the mechanism of action (e.g., lymphocyte depleting activities) of anti-CD52 antibodies in vivo.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention. All publications and other references mentioned herein are incorporated by reference in their entirety. In case of conflict, the present specification, including definitions, will control. Although a number of documents are cited herein, this citation does not constitute an admission that any of these documents forms part of the common general knowledge in the art. Throughout this specification and claims, the word “comprise,” or variations such as “comprises” or “comprising” will be understood to imply the inclusion of a stated integer or group of integers but not the exclusion of any other integer or group of integers. The materials, methods, and examples are illustrative only and not intended to be limiting.

### EXAMPLES

The following examples are meant to illustrate the methods and materials of the present invention. Suitable modifications and adaptations of the described conditions and parameters normally encountered in the art that are obvious to those skilled in the art are within the spirit and scope of the present invention.

In Examples 1-8, we describe the generation of a human CD52 transgenic mouse (also referred as “hCD52 transgenic mouse”) model and our use of it to investigate the mechanism of action of alemtuzumab (Hu et at, Immunology 128:260-270 (2009)). Eight- to twelve-week old heterozygous hCD52 transgenic mice were used unless otherwise specified. In these examples, data were analyzed using GraphPad Prism V4.03 (GraphPad Software, San Diego, Calif., USA). Comparisons for multiple groups were made using one way ANOVA with the Dunnett's post hoc test. Differences were considered statistically significant if the p-value was less than 0.05.

In Examples 9-14, we describe studies in xenograft tumor models further supporting the involvement of neutrophils and NK cells in the lymphocyte-depleting activity of alemtuzumab (Sider et al., Leak Lymphoma Epub. (2010)). In these examples, Kaplan-Meier survival analysis was used to compare survival in the different animal groups.

In Examples 15-17, we describe studies that investigated the impact on lymphocyte depletion by co-administering alemtuzumab (CAMPATH®-1H) with G-CSF (NEUPOGEN®) or co-administering alemtuzumab with GM-CSF (LEUKINE®), in a hCD52 transgenic mouse model. In those studies, we also evaluated the kinetics of lymphocyte depletion in response to co-administration of alemtuzumab and G-CSF.

### Example 1

### Generation of Transgenic Mice Expressing hCD52

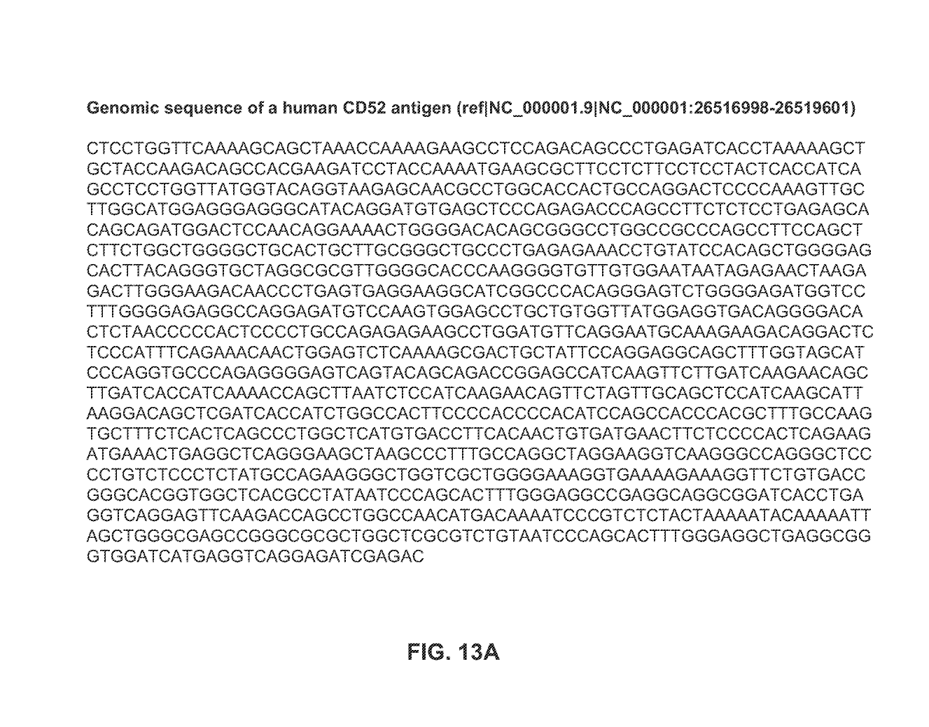

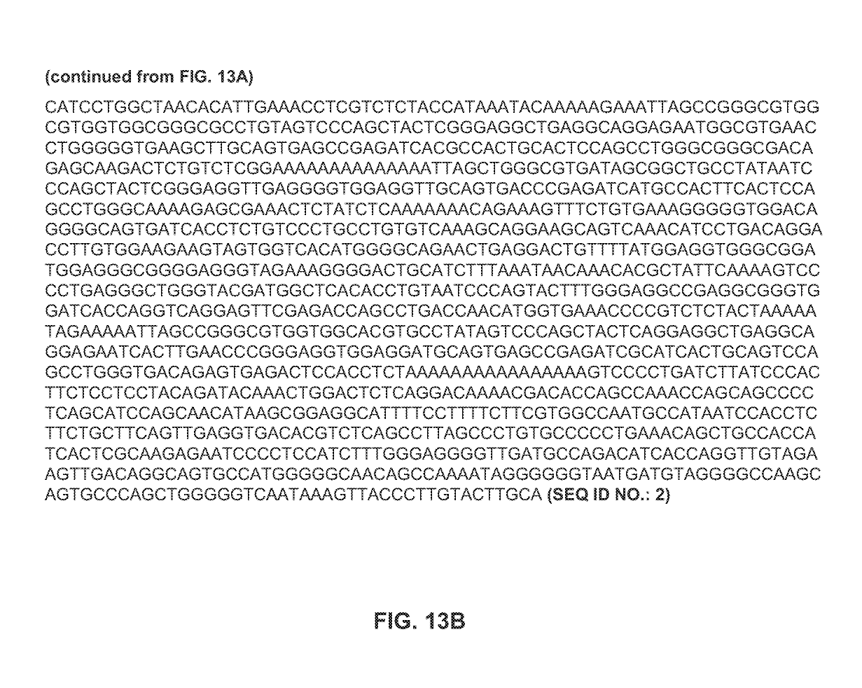

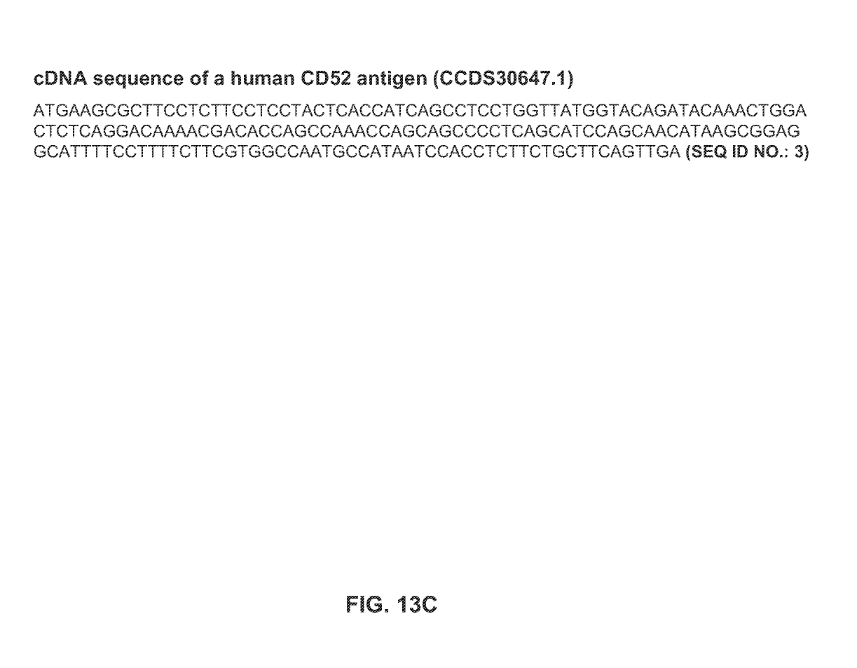

The transgenic mouse expressing hCD52 was created on a CD1 mouse strain background at Xenogen Biosciences (Cranbury, N.J.) by microinjecting mouse embryonic stem cells with a bacmid construct consisting of about 145 kb of genomic DNA from human chromosome 1 containing the entire hCD52 gene and promoter sequence (NCBI MIM: 114280; GeneID: 1043; see FIGS. 13A-C). The murine CD52 gene remained present. Genetic determination of homozygosity or heterozygosity in hCD52 transgenic mice was performed on tail clips using polymerase chain reaction (PCR). Homozygous or heterozygous hCD52 transgenic mice were found to have a normal physical appearance, physiological activities, body weights, and life span comparable to the wild type CD1 background strain.

### Example 2

### Tissue Distribution of hCD52 in Transgenic Mice Expressing hCD52

### A. Immunohistochemistry Assays

Formalin-fixed, paraffin-embedded samples of spleen, inguinal lymph nodes and associated adipose tissue, thymus, pancreas, stomach, testes, ovary and bone/bone marrow from six heterozygous hCD52 transgenic mice (3 males and 3 females) and two CD1 wild-type mice (1 male, 1 female) were cut into 5 μm sections and stained with hematoxylin and eosin (H&E). Serial sections were assessed for tissue morphology and expression of hCD52 by staining with a monoclonal rat anti-hCD52 antibody (CAMPATH®-1G clone YTH34.5, Serotec, Bavaria, Germany) at a dilution of 1:8000. A rabbit anti-rat secondary antibody (Vector Laboratories, Burlingame, Calif.) was then added at a dilution of 1:250. Detection of positive cells was performed using a biotin-free horseradish peroxidase and polymer detection kit (Mach-2 HRP Rabbit, Biocare, Concord, Calif.) followed by a diaminobenzidine chromogen (Dako, Carpenteria, Calif.), All tissue sections were evaluated qualitatively for staining intensity and distribution by a board certified veterinary pathologist.

### B. Results

Histological evaluation of tissues from hCD52 transgenic mice showed that the morphology of tissues, including the spleen, inguinal lymph nodes and associated adipose tissue, thymus, bone/bone marrow, pancreas, stomach, testes, and ovary was normal and comparable to that observed in CD1 wild-type control mice, indicating that expression of the human transgene product did not affect normal tissue architecture. Staining of the tissues for hCD52 expression revealed a tissue distribution similar to that seen in humans with high levels of expression in lymphoid tissues and positive scattered mononuclear cells in the stomach, testes and adipose tissue. The proximal epithelium and mature sperm in the epididymis stained positive for hCD52, as in humans. Staining was also observed in some granulosa cells and cumulus oophorous cells in the ovary. No hCD52 staining was detected in any of the tissues from CD1 wild-type mice as expected from the absence of hCD52 in these mice and the lack of cross-reactivity of the detecting antibody for mouse CD52. Staining with a control IgG antibody also failed to generate a signal in either wild type or hCD52 transgenic mice.

### Example 3

### Expression of hCD52 in Immune Cell Populations

The pattern of expression and density of hCD52 antigen on various immune cell populations of hCD52 transgenic mice was examined by flow cytometry.

### A. Quantification of CD52 Antigen Density on the Cell Surface

The number of hCD52 molecules present on the surface of different cell populations from hCD52 transgenic mice was quantified using Quantum Simply Cellular anti-human IgG beads from Bangs Laboratories Inc (Fishers, Ind.) according to manufacturer's instructions. Briefly, beads coated with different amounts of anti-human IgG corresponding to a pre-calibrated antibody-binding capacity (ABC) were incubated with saturating concentrations of alemtuzumab/CAMPATH®-1H (Genzyme Corporation, Cambridge, Mass.) labeled with FITC to generate an ABC standard curve based on mean fluorescence intensity (MFI). The ABC standard curve was then used to convert the MFI value obtained with HTC-alemtuzumab binding to a given flow cytometry-delineated subpopulation into the number of hCD52 molecules per cell.

### B. Pattern of CD52 Antigen Expression in Immune Cell Populations

Cell staining was performed by incubating 4×10⁵ to 2×10⁶ cells with FITC-conjugated alemtuzumab/CAMPATH®-1H and fluorescently labeled antibodies specific for mouse cell surface markers including B220 (clone RA3-6B2), CD19 (clone 1D3), CD3 (clone 145-2C11), CD4 (clone RM4-5), CD8 (clone 53-6.7), P480 (clone BM8), CD11b (clone MI/70), GR-1 (clone RB6-8L5), NK1.1 (clone PK136), CD49b (clone DX5), CD44 (clone 1M7), and CD25 (clone PC61.5) purchased from BD Bioscience (San Jose, Calif.) or eBioscience (San Diego, Calif.). T regulatory cell identification was performed by intracellular staining for FoxP3 (clone FJK-16S) as indicated by the manufacturer (eBioscience). Stern cell identification was performed by staining cells isolated from the bone marrow with Mouse Lineage Antibody cocktail (BD Bioscience) simultaneously with Thy-1.1 (clone H1S51), Sca-1 (clone D7), and c-Kit (clone 2B9). Staining of peripheral blood cells was performed by staining 50 μl of whole blood from individual mice with the antibodies described above followed by removal of red blood cells using FACS lysis solution (BD Bioscience) as described by the manufacturer. Fluorescence intensities were measured using either a FACS Calibur or LSR-II (BD Bioscience) and analysis was performed using FlowJo Software (Tree Star Inc., Oreg.). For quantification of absolute numbers of specific cell populations in the peripheral blood, COUNT BRIGHT™ Absolute Counting Beads (Invitrogen, Carlsbad, Calif.) were added to blood samples according to the manufacturer's instructions. For lymphoid organs, the absolute number of cells in a given population was obtained by multiplying the percentage of FACS positive cells by the total number of cells recovered from the organ.

### C. Results

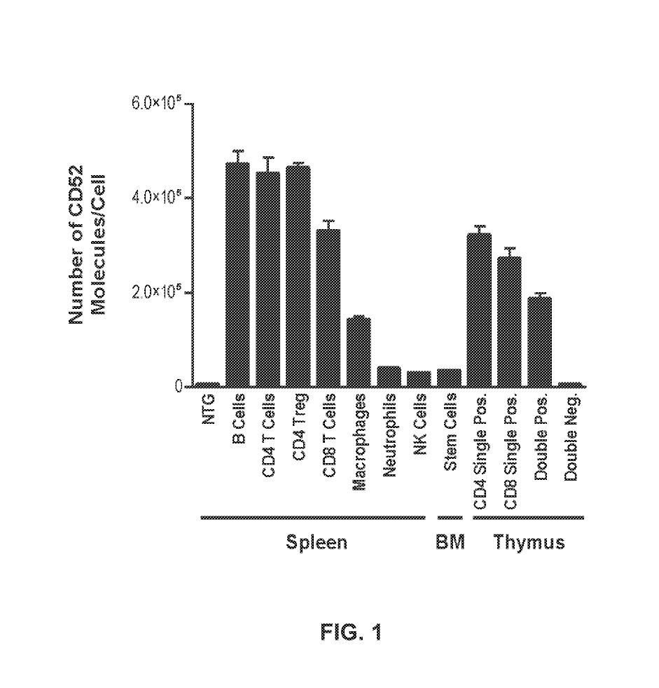

The pattern of hCD52 expression in transgenic mice we observed was similar to that reported in humans (e.g., Hale G. J Biol Regul Homeost Agents 15:386-391 (2001); Huh et al., Blood 92, Abstract 4199 (1998); Elsner et al., Blood 88, 4684-4693 (1996); Gilleece et al, Blood 82, 807-812 (1993); Rodig et al., Clin Cancer Res 12: 7174-7179 (2006); Ginaldi et al., Leuk Res 22: 185-191 (1998); Domagala et al., Med .Sci Monit 7:325-331 (2001)). The highest levels of expression were seen on B and T lymphocytes with lower levels on macrophages and little or no expression on mature NK cells, neutrophils and bone marrow stern cells (FIG. 1), The actual number of hCD52 molecules per lymphocyte (3.3×10⁵ for CD8⁻ T cells, 4.7×10⁵ for CD4⁺ T cells and B cells) was also comparable to the number found on unfractionated human peripheral mononuclear cells (4.2−4.5×10⁵) as described in Bindon et at, Eur J Immunol 18:1507-1514 (1988). CD52 expression in the thymus has not been previously studied in humans, but our analysis of thymic cells from hCD52 transgenic mice indicated that robust levels of hCD52 were present on CD4/CD8 single positive as well as double positive thymocytes with no detectable expression above background on double negative thymocytes (FIG. 1).

### Example 4

### Evaluation of Immune Status of hCD52 Transgenic Mice

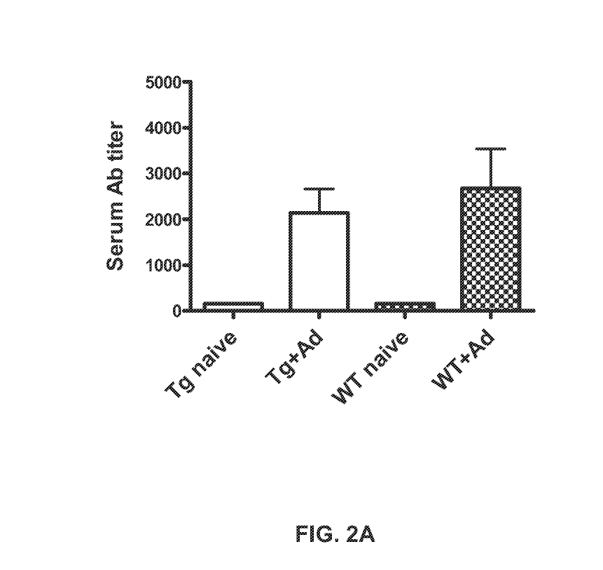

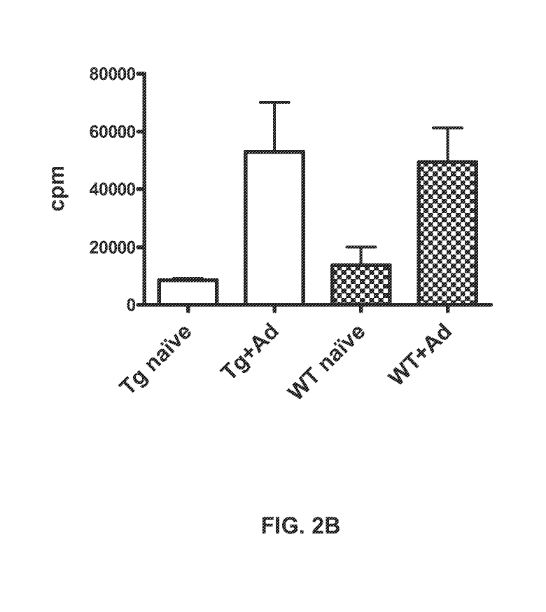

To assess the immune status of hCD52 transgenic mice compared to wild type CD1 mice, three wild type CD1 mice and three hCD52 transgenic CD1 mice were immunized intradermally with 1×10⁹ infectious units of a non-replicating E1-deleted adenovirus (Ad) serotype 2 vector lacking a transgene. Three weeks later, serum samples and spleens were collected from individual mice to assess humoral and cellular immune responses to Ad. Titers of antibodies to Ad were measured by ELISA as described in Kaplan et al., Hum Gene Ther 8:1095-1104 (1997). Briefly, 2-fold serial dilutions of serum were added to wells coated with inactivated Ad2 particles and bound Ad-specific antibodies were detected by the addition of HRP-conjugated goat anti-mouse IgG, A, M (Cappel, Durham, N.C.) followed SigmaFAST OPD substrate (Sigma, St. Louis, Mo.), The serum titer was defined as the reciprocal of the highest dilution of serum producing a colorimetric signal with an optical density ≦0.1. The cellular immune response was measured by stimulating spleen cells (5×10⁵/well of 96-well plate) with inactivated Ad particles (1 μg/ml) for 5 days, as described in Kaplan et al., Hum Gene Ther 8:1095-1104 (1997). The amount of proliferation induced was measured by pulsing with 1 μCi/well ³H-thymidine for the last 18 hours of incubation. Results shown are mean ±SEM of the values obtained with individual mice. As shown in FIGS. 2A and 2B, transgenic and wild type mice developed comparable levels of Ad-specific antibodies (FIG. 2A) and splenocytes from the animals displayed equivalent levels of proliferation when stimulated with Ad antigen in vitro (FIG. 2B). These results indicate that immune function was not compromised by the expression of hCD52 in the transgenic mice.

### Example 5

### Immune Cell Depletion Following Treatment with Alemtuzumab

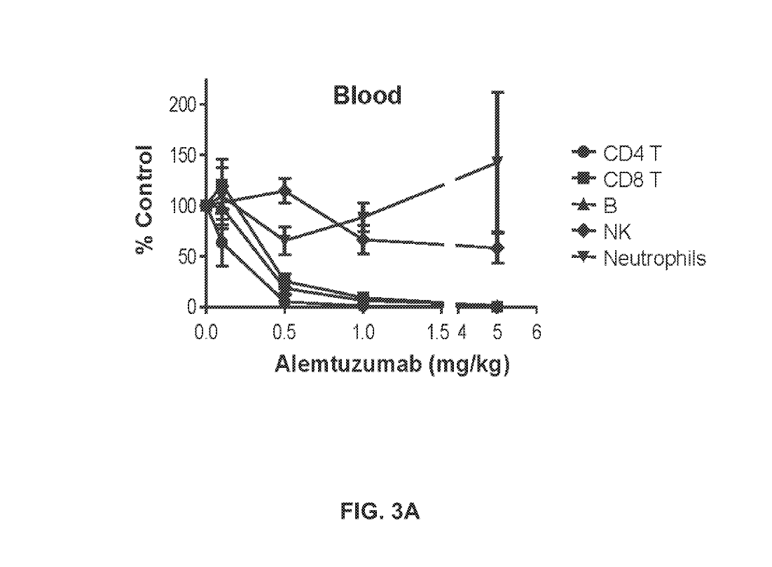

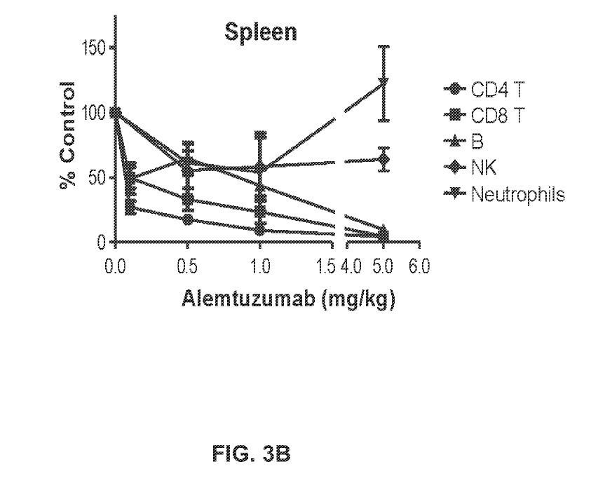

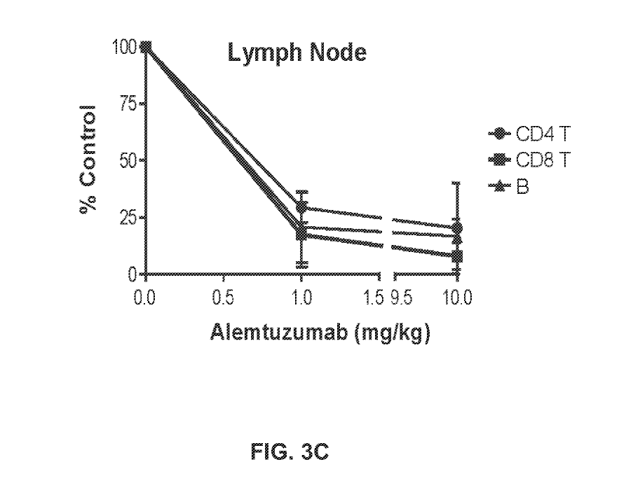

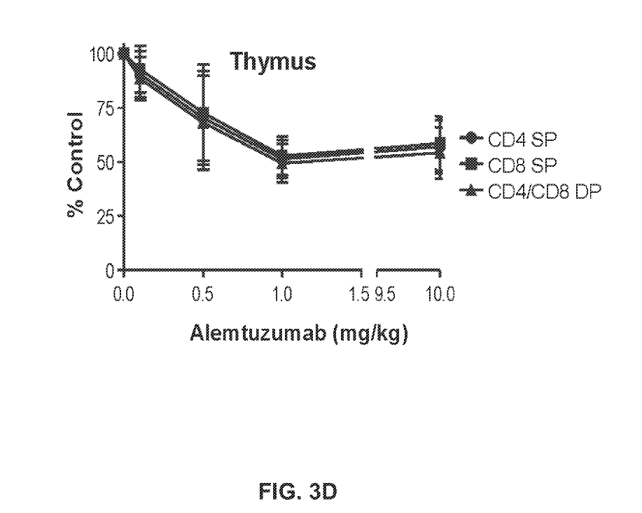

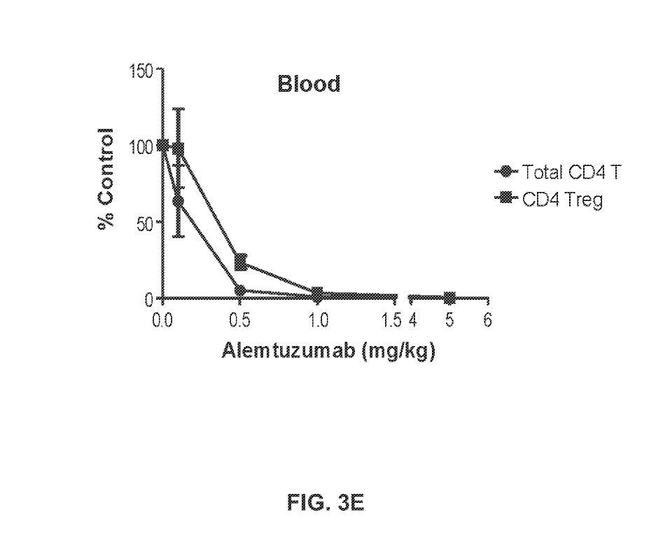

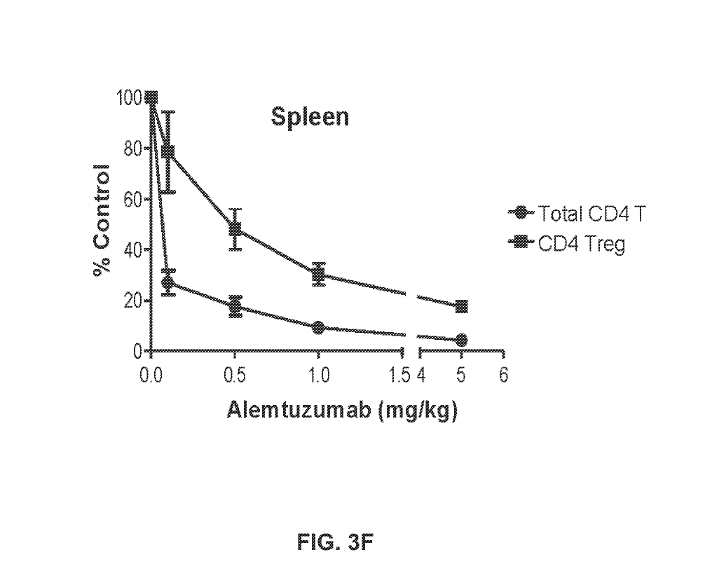

Human CD52 transgenic mice were treated with a single intraperitoneal (i.p.) injection of alemtuzumab at the doses as indicated in FIGS. 3A-3F or with phosphate buffered saline (PBS) as a vehicle control. Blood and lymphoid organs from individual mice (n=5) were collected at 72 hours post treatment for flow cytometry analysis. As observed in humans (Brett et al., Immunology 88:13-19 (1996); Cox et al., Eur J Immunol 35:3332-3342 (2005); Coles et al., N Engl J Med 359:1786-801 (2008); Buggins et al., Blood 100:1715-20 (2002); Buggins et al., Blood 100:1715-1720 (2002)), treatment with doses of alemtuzumab ≧1 mg/kg (doses comparable to those administered to multiple sclerosis and rheumatoid arthritis patients) resulted in a near complete depletion of B and T lymphocytes from the circulation (FIG. 3A), while mature NK cells and neutrophils which express little CD52 were not as affected (FIG. 3A). There was a trend for increased numbers of neutrophils in the blood and spleen at the highest dose of alemtuzumab (FIGS. 3A and 3B). This effect could involve factors such as recruitment of neutrophils from the bone marrow or demarginalization.

The degree of lymphocyte depletion was not as profound in lymphoid organs, an assessment that has not been possible to conduct in humans. For example, at doses of 0.5-1 mg/kg alemtuzumab, most lymphocytes were depleted from the circulation but significant numbers of B and T cells were still present in the spleen, lymph nodes, bone marrow and thymus as determined by flow cytometry analysis (FIGS. 3A-3D) and histochemistry (data not shown). A higher dose of 5 mg/kg was required to achieve a more complete depletion in the spleen while depletion was still incomplete in the lymph nodes and thymus even at 10 mg/kg. In particular, a maximum depletion of only ˜50% of single and double positive thymocytes was achievable in the thymus even though the level of CD52 expression by these cells is quite robust (FIG. 1). This may have been due to reduced penetrance and exposure to alemtuzumab and/or less robust ADCC effector system in the lymphoid organs.

A relative sparing of T cells with a regulatory phenotype (CD4⁺CD25⁺FoxP3⁺) compared to total CD4⁺ T cells was observed in both the blood and spleen (FIGS. 3E and 3F) even though both populations express equivalent levels of CD52 on their surface (FIG. 1).

### Example 6

### Pattern of Lymphocyte Repopulation After Treatment with Alemtuzumab

The kinetics of peripheral blood lymphocyte repopulation has been described in multiple sclerosis patients treated with alemtuzumab (Coles et al., J Neurol 253:98-108 (2006); Cox et al., Eur J Immunol 35:3332-3342 (2005); Coles et al. N Engl Med 359:1786-1801 (2008)). In these patients, after near complete depletion from the circulation, B lymphocytes return to pre-treatment levels between 3 and 6 months while cell counts rise slowly and remain below normal for several years. We used the hCD52 transgenic mice to study pattern of lymphocyte repopulation. Human CD52 transgenic mice were treated with a single intraperitoneal (i.p.) injection of alemtuzumab at a single 10 mg/kg dose of alemtuzumab or with PBS as a vehicle control. Serial blood collections were performed on individual mice (n=8) at 72 hours and at various intervals out to 25 weeks post treatment. Results are shown as “(% Control,” which were calculated by taking the absolute number of cells in a given cell population in individual alemtuzumab-treated mice and dividing it by the mean absolute number of the same population in control mice.

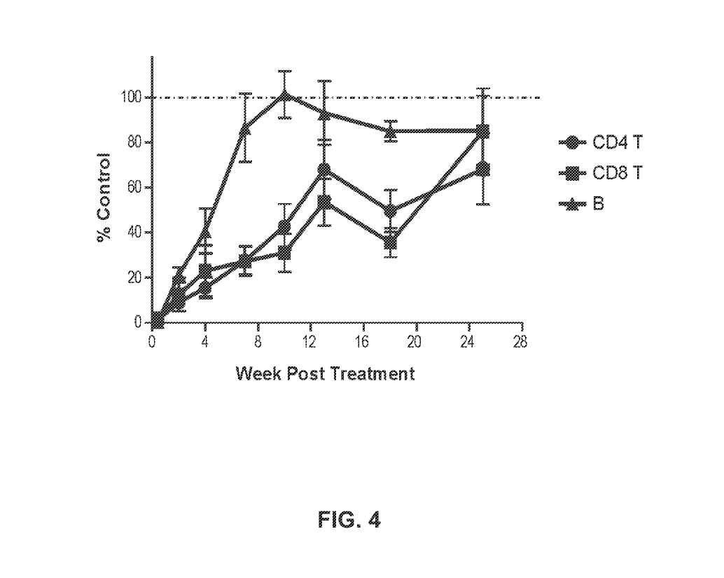

We observed a similar repopulation pattern in the hCD52 transgenic mice, albeit in a more contracted time frame. Treatment of alemtuzumab resulted in essentially complete depletion of B and T lymphocytes from the circulation. B lymphocytes returned to baseline levels by 7-10 weeks post treatment while CD4⁺ and CD8+ T cells recovered more slowly and were still below normal levels at 25 weeks (FIG. 4). Treatment with alemtuzumab did not significantly affect the bone marrow (data not shown) or deplete CD52-negative hematological precursors, which might allow for the rapid recovery of B lymphocytes. Examination of the thymus has not been possible in humans but, in the transgenic mouse, we observed partial depletion of single and double positive thymocytes, reaching a maximum of approximately 50% at the highest dose of 10 mg/kg of alemtuzumab in Example 3 (FIG. 3D). This partial loss of thymocytes may account in part for the slower recovery of T lymphocytes.

### Example 7

### Studies on Mechanism of Lymphocyte Depletion by Alemtuzumab

Alemtuzumab has been reported to mediate lysis of CD52⁺ cells in vitro via complement-dependent cytotoxicity (CDC) and antibody-dependent cell-mediated cytotoxicity (ADCC) (Lowenstein et al., Transplant Intl 19:927-936 (2006)). However, the relative contribution of these mechanisms in vivo remains undefined. The hCD52 mice offered a unique opportunity to explore this issue by selectively removing each effector mechanism and assessing the impact on the depleting activity of alemtuzumab.

Mice were treated to remove selected effector arms of the immune system to study the impact on the cytokine induction and lymphocyte-depleting activity of alemtuzumab. Complement was inactivated by treatment with cobra venom factor (Calbiochem, San Diego, Calif.) administered i.p. at 1 mg/kg at 72 and 24 hours prior to the administration of alemtuzumab. Depletion of complement was confirmed to be 85-90% using an ELISA kit to measure C3 according to manufacturer's instructions (Immunology Consultants Laboratory Inc, Newberg, Oreg.). NK cells were removed by treatment with anti-asialo-GM1 antibody (Wake Chemicals USA, Inc., Richmond, Va.) administered i.v. at 25 mg/kg, 72 and 24 hours prior to the administration of alemtuzumab. Neutrophils were depleted with anti-Gr-1 antibody (anti-Ly-6G, eBioscience, San Diego, Calif.) given i.v. at 7.5 mg/kg, 72 and 24 hours prior to the injection of alemtuzumab. Depletion of NK cells and neutrophils from the blood was confirmed by flow cytometry and was found to be 85-90% and 95%, respectively.

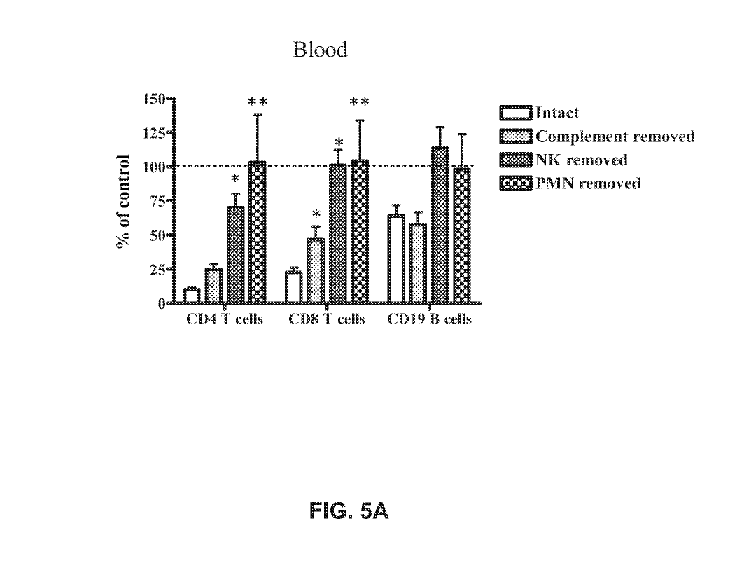

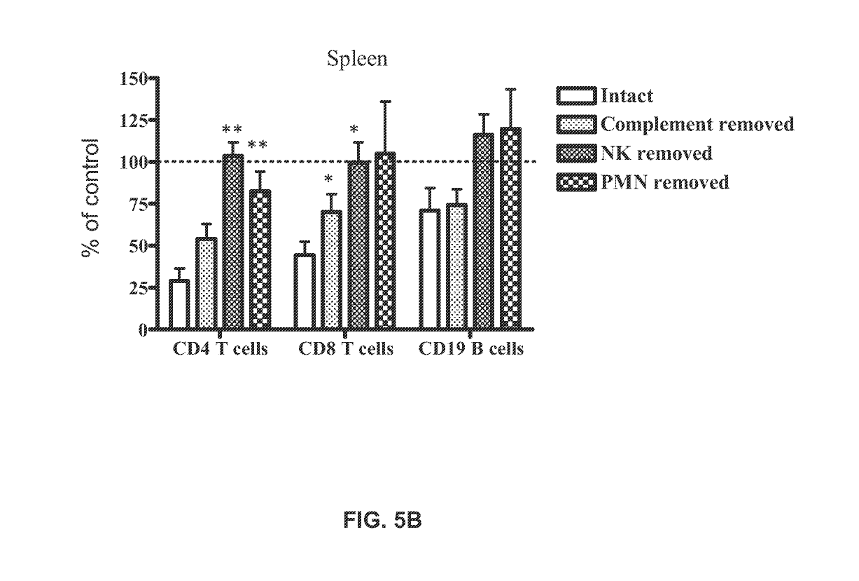

As shown in FIGS. 5A and 5B, removal of complement with cobra venom factor had little or no impact on the depletion of blood or splenic B and T lymphocytes by alemtuzumab. In contrast, removal of NK cells with an anti-asialo GM-1 antibody or neutrophils with an antibody against Gr-1, significantly reduced or ablated the activity of alemtuzumab suggesting a predominant role for ADCC as opposed to CDC in lymphocyte depletion. The involvement of neutrophils as effectors in the activity of alemtuzumab was a novel finding.

### Example 8

### Induction of Serum Cytokines by Alemtuzumab

Administration of alemtuzumab in humans results in an infusion reaction associated with the induction of serum cytokines including TNF-α, IL-6 and interferon-γ (Brett et al., Immunology 88:13-19 (1996); Coles et al., J Neurol 253:98-108 (2006); Coles et al. N Engl J Med 359:1786-1801 (2008); Coles et al., The Lancet 354:1691-1695 (1995); Wing et al., J Clin Invest 98:2819-2826 (1996)). The mechanism of induction and source of the cytokines are not fully characterized. We studied cytokine induction in the hCD52 transgenic mouse. The transgenic mice were given a single i.p. administration of antibody at the doses indicated and serum was collected from individual mice at 1, 2, 4 and 24 hours post dosing (n=5-7). REMICADE® (Genentech Inc, San Francisco, Calif.), a human IgG1 monoclonal antibody against human TNF-α which does not cross-react with mouse TNF-α, was used as a negative control. Serum cytokine concentrations were determined using a BD Cytometric Bead Array (Mouse Inflammation kit; BD Biosciences, San Diego, Calif.) according to manufacturer's protocol.

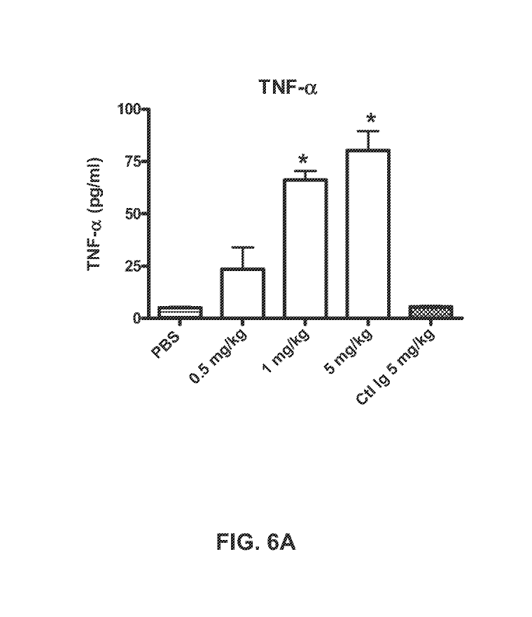

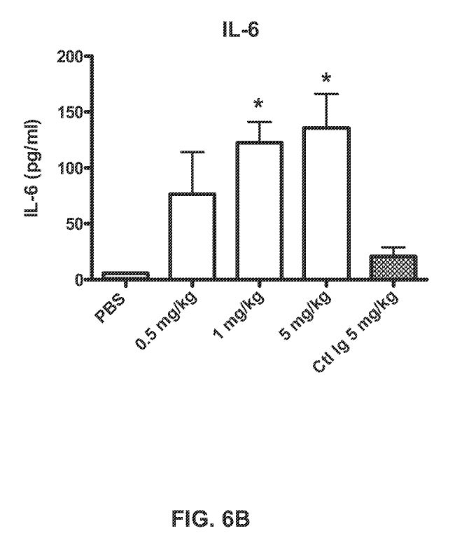

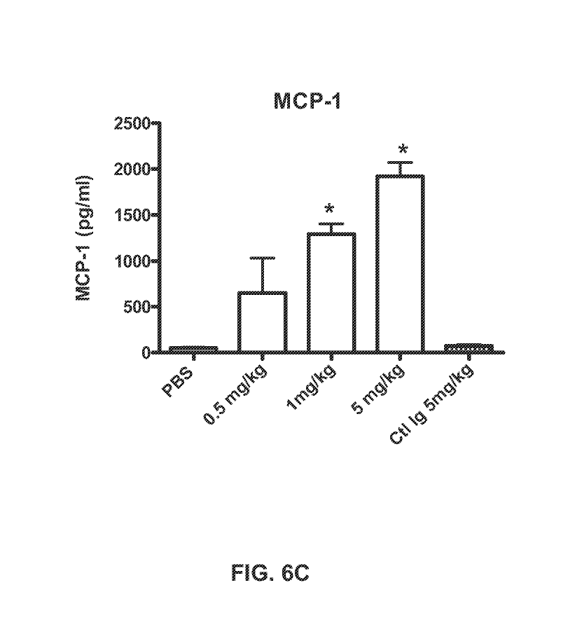

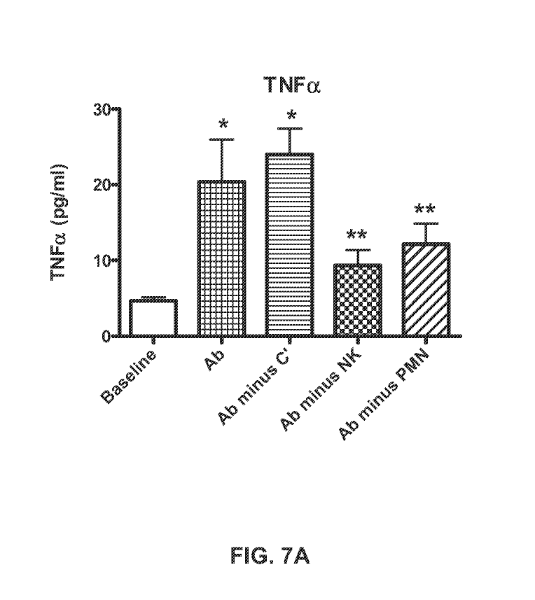

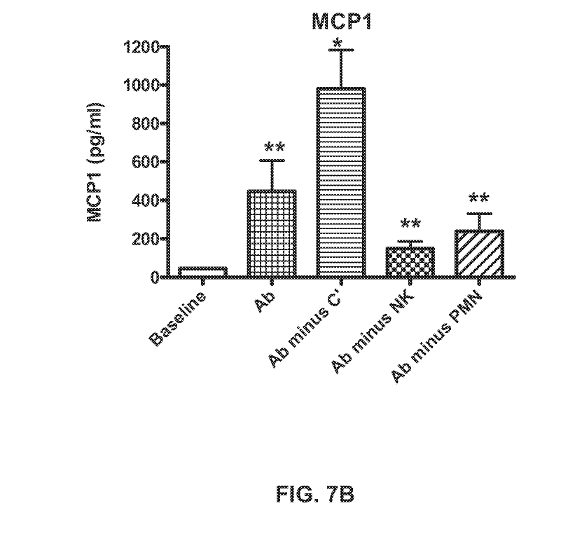

A dose-dependent cytokine peak, including TNF-α, IL-6 and MCP-1, was observed at 2 hours post-injection (FIGS. 6A-6C), followed by a return to basal levels by 24 hours (data not shown). The mechanism responsible for cytokine release was further investigated. Removal of complement did not significantly affect cytokine induction by alemtuzumab (FIGS. 7A and 7B). Removal of NK cells by treatment with anti-asialo GM-1 or neutrophils by treatment with anti-Gr-1, showed an equivalent and significant dampening of cytokine induction (FIGS. 7A and 7B). These results suggest that lymphocyte depletion and cytokine induction are linked processes involving effector cells rather than complement activation.

The following examples describe our studies of alemtuzumab activity in xenograft tumor models.

### Example 9

### Quantitation of CD52 Antigen in Tumor Cell Lines

### A. Cell Lines

The following tumor cell lines were used: B104 (non-Hodgkin's burkitt lymphoma line); Raji (non-Hodgkin's Burkitt lymphoma lines); MC/CAR (multiple myeloma line); Ramos (Burkitt lymphoma line); IM-9 (B lymphoblast line). These tumor cell lines were purchased from the American Type Culture Collection (Manassis, Va.). Cells were grown in the recommended media supplemented with 110% fetal calf serum, 100 units/mi penicillin, 100 units/ml streptomycin and 2 mM glutamine. Each cell line was kept in culture for no more than 25 passages at which time a new vial of cells was thawed. In addition, all cell lines were confirmed to be mycoplasma and pathogen free.

A cell line stably expressing high levels of hCD52 was generated and used in the following examples. CHO-K parental cells (ATCC) were transduced with a plasmid encoding the full-length hCD52 protein along with a neomycin resistance gene. Cells were grown in neomycin-containing medium. Neomycin-resistant single cell clones were isolated by limiting dilution and screened for high levels of CD52 expression, using a FITC-conjugated version of alemtuzumab (Genzyme Corporation, Cambridge, Mass.). In vivo characterization of the final stable cell line indicated that the presence of the plasmid did not affect its ability to form tumors in severe combined immunodeficient (KID) mice nor altered its growth kinetics compared to the unmodified parental cell tine.

### B. Flow Cytometry Analysis of CD52 Antigen Expression

The above-identified tumor cell lines were assessed for the presence and density of CD52 antigen expression by flow cytometry analysis. The number of hCD52 molecules present on the surface of different cell lines was quantified using Quantum Simply Cellular anti-human IgG beads from Bangs Laboratories Inc (Fishers, Ind.) according to manufacturer's instructions. Briefly, beads coated with different amounts of anti-human IgG corresponding to a pre-calibrated antibody-binding capacity (ABC) were incubated with saturating concentrations of FITC-labeled alemtuzumab to generate an ABC standard curve based on mean fluorescence intensity (MFI). The ABC standard curve was then used to convert the MFI value obtained with FITC-alemtuzumab binding to tumor cells into the number of hCD52 molecules per cell.

### C. Results

The non-Hodgkin's Burkitt lymphoma lines B104 and Raji expressed homogeneously high levels of CD52 (358,000 and 458,000 molecules/cell, respectively). By comparison, the Ramos Burkitt lymphoma line showed heterogeneous CD52 expression containing both a negative/low expressing population and a high expressing population (366,000 molecules/cell) The MC/CAR multiple myeloma line also displayed high levels of CD52 (107,000 molecules/cell), while the IM-9 B lymphoblast line expressed minimal levels of the antigen (7,000 molecules/cell). A CHO cell line engineered to stably express CD52 displayed the highest levels of CD52 antigen with 840,000 molecules/cell.

### Example 10

### Activity of Alemtuzumab in Disseminated Tumor Models

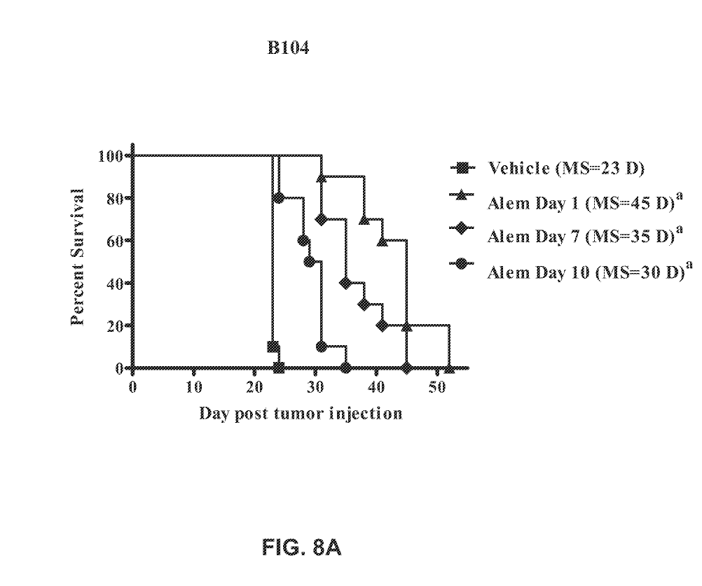

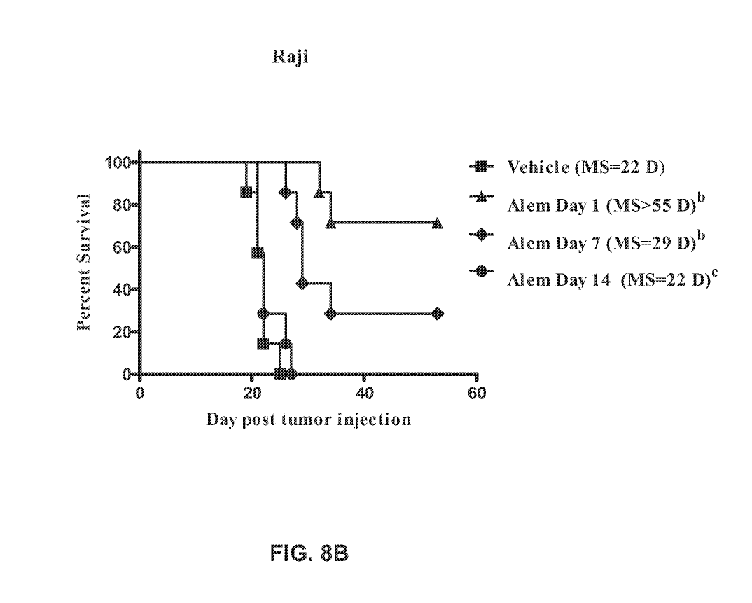

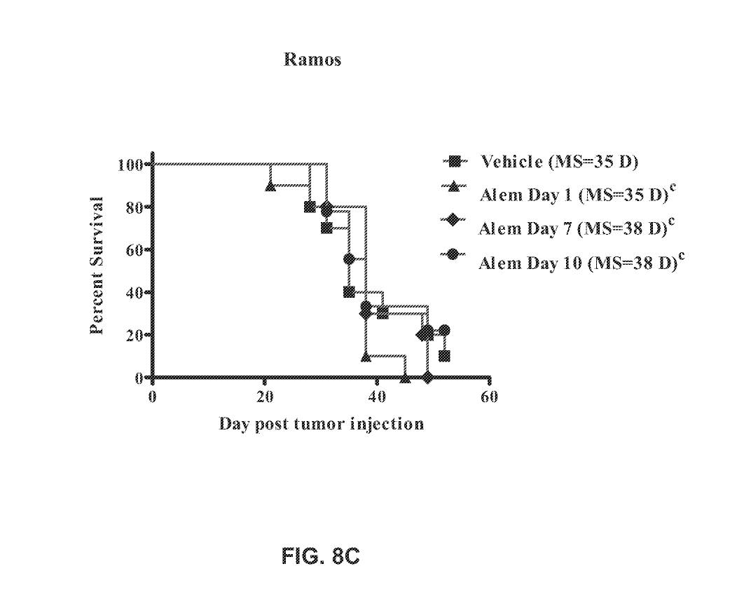

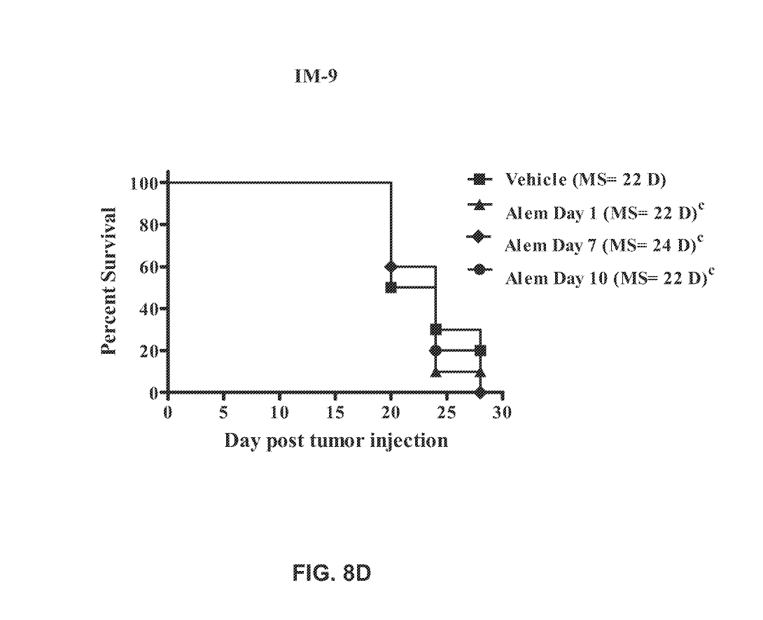

The anti-tumor activity of alemtuzumab against the CD52-expressing cell lines described in Example 9 was explored in a disseminated tumor setting. Six- to eight-week old female SCID mice were purchased from Charles River Laboratories (Wilmington, Mass.). Animal experiments were approved by Genzyme Institutional Animal Care and Use Committee and performed according to the standards of the association for Assessment and Accreditation of Laboratory Animal Care. Cells (100 μl) from the B104, Raji, Ramos and IM-9 tumor cell lines were injected intravenously at a concentration predetermined to be optimal, into the tail vein of SCID mice. In each case, seeding of the tumor cells resulted in involvement of the central nervous system and gave rise to hind limb paralysis (Hernandez-Ilizaliturri et al., Leuk Lymphoma 46:1775-1784 (2005); Lapalombella et al., Clin Cancer Res 14:569-578 (2008); Hernandez-Ilizaliturri et al., Clin Cancer Res 9:5866-5873 (2003); de Kroon JFEM et al., Exptl Hematol 24:919-926 (1996); Carlo-Stella et al., Exptl Hematol 34:721-727 (2006)). Treatment with alemtuzumab was initiated at various time points post-tumor cell injection (day 1-day 21) and consisted of twice weekly i.p. injections of 10 mg/kg (FIGS. 8A-8D). Kaplan-Meier survival analysis was performed using the GraphPad Prism version 4.03 software (San Diego, Calif., USA). Data were considered statistically significant if the p-value was less than 0.05. All in vivo experiments shown were repeated at least twice.

As observed in the B104 and Raji models (FIGS. 8A and 8B), antibody treatment was most efficacious if initiated on day 1 when tumor growth was the least advanced, resulting in the most sizeable increase in survival. Alemtuzumab was also capable of therapeutic activity against established tumors and provided a reduced but significant increase in survival in the B104 model when the start of treatment was delayed until day 7 or 10 post-tumor cell injection, and in the Raji model when treatment was delayed until day 7 but not longer (FIGS. 8A and 8B). In contrast to these results, alemtuzumab failed to control the growth of disseminated Ramos tumor cells even when treatment was initiated on day 1 post-tumor injection (FIG. 8C). As described in Example 9, flow cytometry analysis indicated that CD52 expression by this tumor line is heterogeneous and it is possible that the lack of efficacy was due to the outgrowth of the CD52 negative population. Alemtuzumab also failed to show any significant activity against IM-9 tumor cells, which display very low levels of CD52 (FIG. 8D), Overall, these results suggest that density of the target CD52 antigen is one important factor in the efficacy of alemtuzumab as the inhibition of tumor growth was greatest against tumor cells expressing homogeneously high levels of CD52. Human B-CLL samples have been described to express levels of CD52 comparable to those of the susceptible Raji and B104 cell lines (371,303 molecules/cell) (Rossmann et al., Hematol J 2:300-306 (2001)), in line with the clinical efficacy of alemtuzumab in this indication. Thus, levels of CD52 expression in cancer patients may be used to predict efficacy of alemtuzumab treatment.

### Example 11

### Activity of Alemtuzumab in Subcutaneous Tumor Models

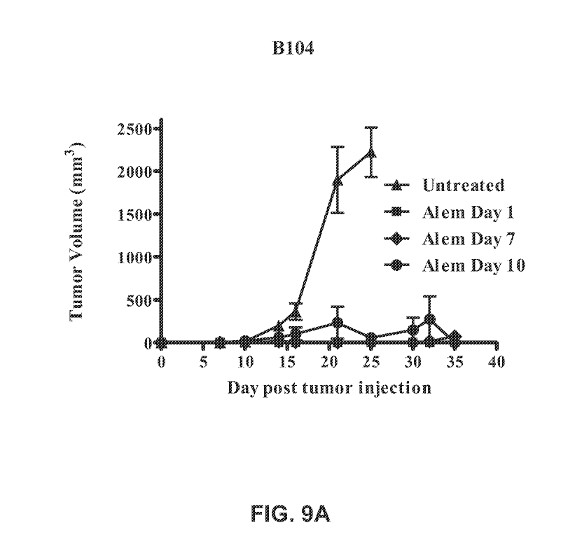

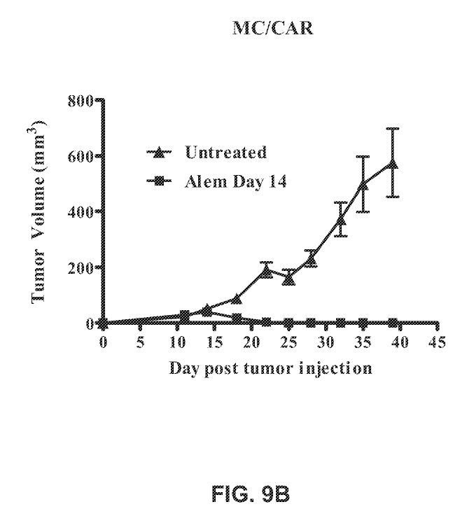

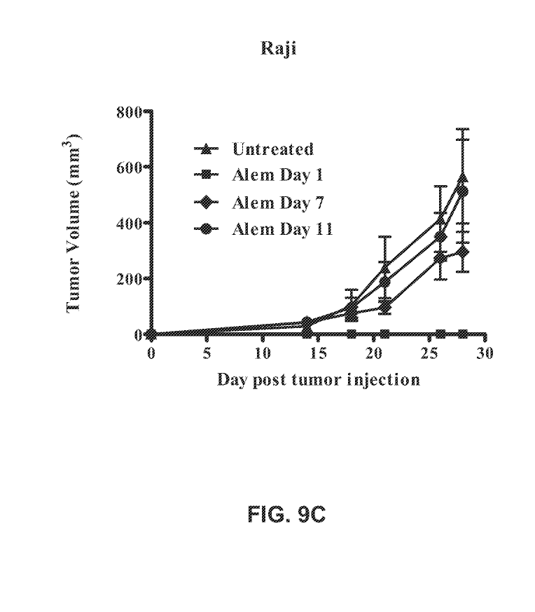

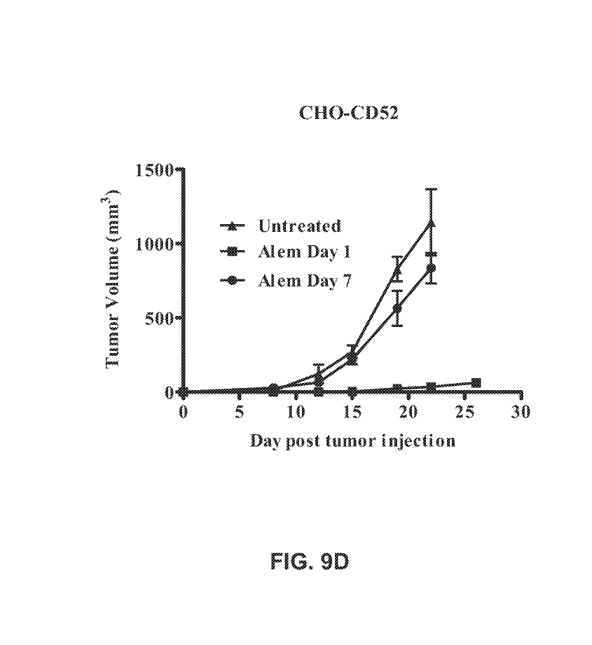

Clinical experience with alemtuzumab indicates that the antibody exerts its greatest activity against tumor cells in the blood and bone marrow and is not as efficacious against bulky disease (Cortelezzi et al., Haematologica 90: 410-412 (2005); Lundin et al., Blood 100:768-773 (2002); Lin et al., Leukemia 19:1207-1210 (2005)). Therefore, we examined the activity of alemtuzumab in the context of solid subcutaneous (s.c.) tumors (FIGS. 9A-9D). Six- to eight-week old female SCID mice were purchased from Charles River Laboratories (Wilmington, Mass.). Animal experiments were approved by Genzyme Institutional Animal Care and Use Committee and performed according to the standards of the association for Assessment and Accreditation of Laboratory Animal Care. To generate subcutaneous (s.c.) tumors, cells from the Raji, B104, MC/CAR and CHO-CD52 lines were resuspended at the desired concentration and 100 μl was injected s.c. into the flank of each mouse. The optimal number of cells required to obtain 100% tumor take was optimized for each tumor line. Tumor size was measured twice a week with electronic digital calipers and animals were sacrificed when tumor size reached ≧1500 mm³. Treatment with alemtuzumab (10 mg/kg i.p., twice per week) was initiated at various time points post tumor cell injection (day 1-day 14). Kaplan-Meier survival analysis was performed using the GraphPad Prism version 4.03 software (San Diego, Calif., USA). Data were considered statistically significant if the p-value was less than 0.05. All in vivo experiments shown were repeated at least twice.

In spite of the uniformly high level of CD52 expression in all lines tested, variable degrees of efficacy were observed. Near complete inhibition of tumor growth was observed in the B104 and MC/CAR models even when initiation of treatment was delayed until day 10 post-tumor injection for B104 (FIG. 9A) and day 14 for MC/CAR, a time at which tumors were palpable (50 mm³; FIG. 9B). By comparison, equivalent inhibition of tumor growth in the Raji (FIG. 9C) and CHO-CD52 (FIG. 9D) models was only seen when antibody treatment was initiated on day 1 post-tumor cell injection and was not observed if treatment was delayed until day 7 post-tumor injection. Therefore, it suggests that other factors, in addition to CD52 antigen density, also play a determining role in the efficacy of alemtuzumab treatment against s.c. tumors. Such factors may include, without being limited to, the aggressiveness of tumor growth, differences in intrinsic survival mechanisms of the tumor cells, and ability of the antibody to penetrate the tumor mass. The alemtuzumab-induced regression of 50 mm³ MC/CAR tumors suggests that the antibody can at least penetrate small tumors (FIG. 9B) resulting in effective inhibition of tumor growth. Specific localization of alemtuzumab into larger CD52 s.c. tumors was also demonstrated histologically in the CHO-CD52 tumor model (data not shown). Alemtuzumab administered i.p. to mice bearing 200 mm³ tumors could be detected 4 hours later on the surface of tumor cells within CHO-CD52⁺ tumors, but was absent in parental CHO CD52⁻ tumors. Treatment with alemtuzumab at this stage of tumor growth was ineffective in spite of its observed ability to penetrate the tumor mass and bind to the surface of tumor cells suggesting that the reduced clinical efficacy of alemtuzumab against tumor masses is unlikely to be solely due to a lack of tumor penetrance.

### Example 12

### Inactivation of Immune Effector Mechanisms Inhibits the Anti-tumor Activity of Alemtuzumab

### A. Deglycosylation of Alemtuzumab Abolishes its Anti-tumor Activity In Vitro of Alemtuzumab

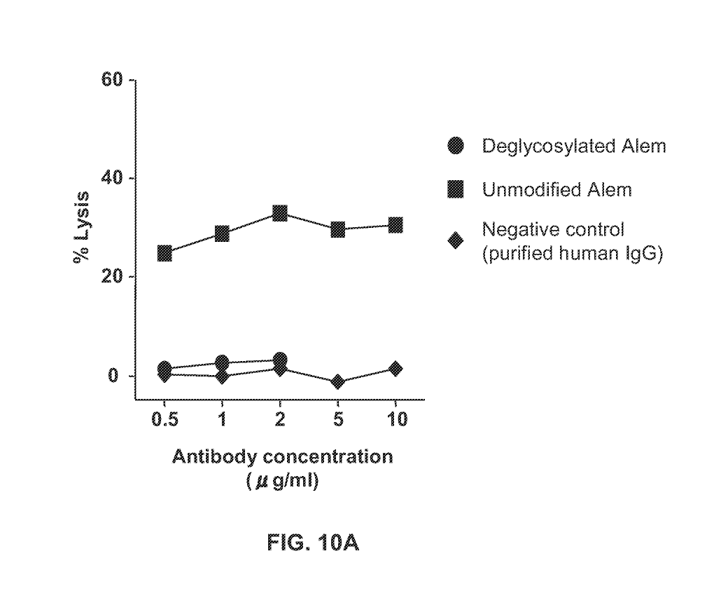

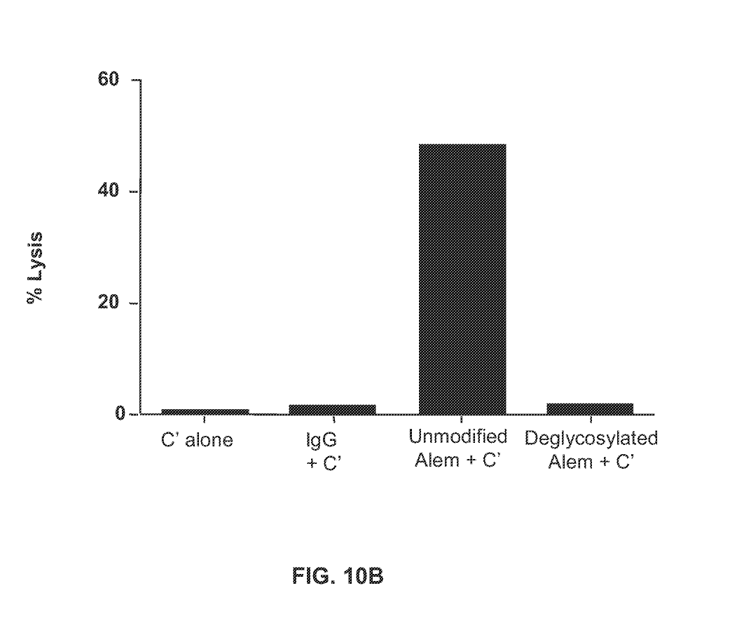

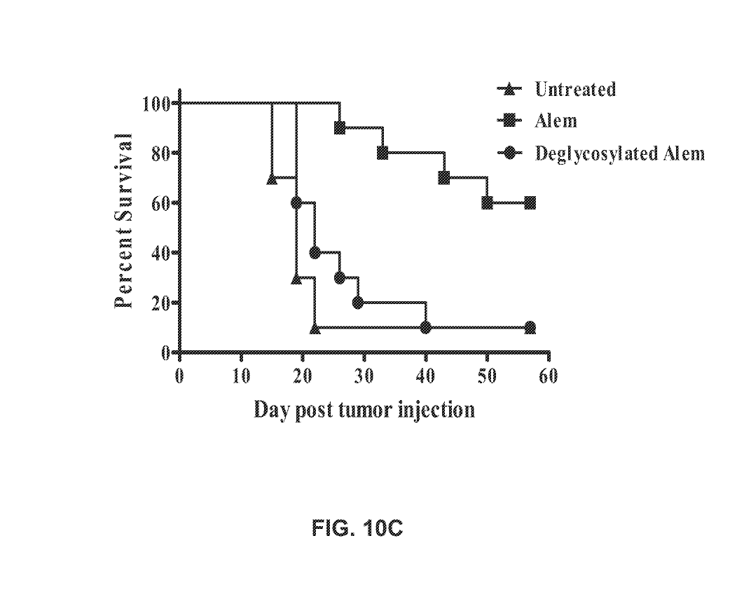

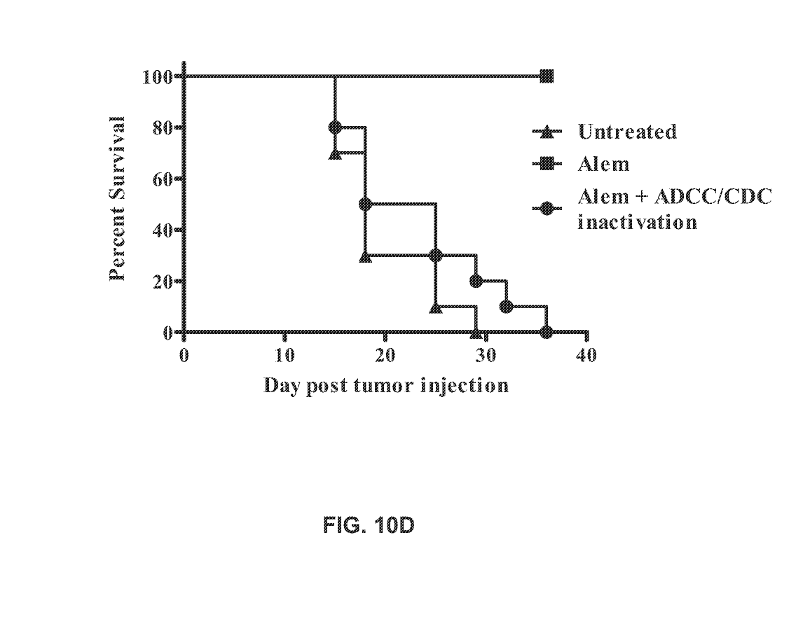

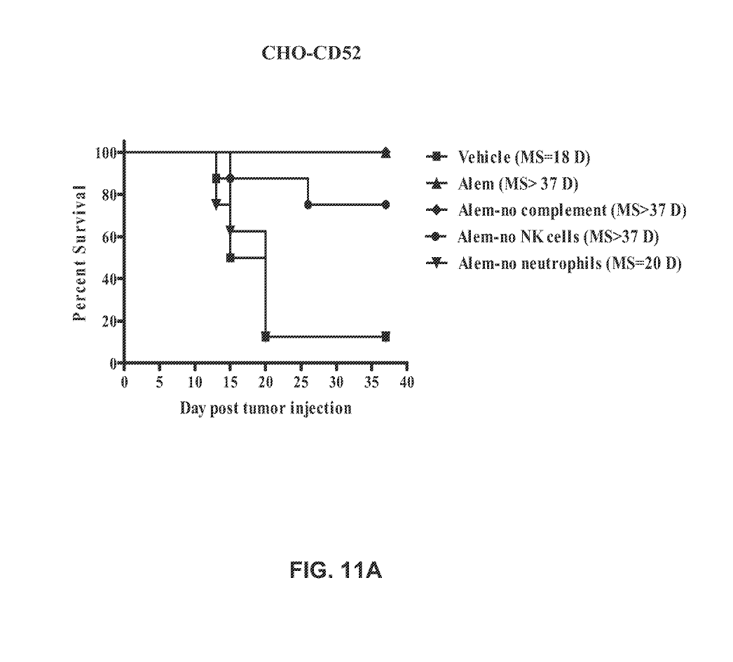

Alemtuzumab is a recombinant humanized IgG1 monoclonal antibody. Antibodies of the human IgG1 isotype are capable of CDC and ADCC mediated by interaction of the Fcγ2 portion with C1q and effector cell Fe receptors, respectively. This interaction involves the contribution of carbohydrates in the Fcγ2 region (Jefferis R et al., Immunol Rev 163:59-76 (1998)). We observed that alemtuzumab displayed robust CDC and ADCC activity against CD52⁺ cells in vitro using CHO-CD52 cells as a target (FIGS. 10A and 10B), as expected from the IgG1 isotype of alemtuzumab (see also Golay et al., Haematologica 89: 1476-1483 (2004); Zent et al., Leuk Res 32:1849-1856 (2008); Cruz et al., Leuk Lymphoma 48:2424-2436 (2007)). We further generated deglycosylated alemtuzumab by treatment with peptide N-glycosidase F in 50 mM sodium phosphate buffer, pH7.0, at 37° C. overnight. Carbohydrate removal was confirmed by sodium dodecyl sulfate polyacrylamide gel electrophoresis (SDS-PAGE), matrix-assisted laser desorption/ionization time-of-flight (MALDI-TOF) mass spectrometry analysis and lectin blotting. Deglycosylation of the antibody removes the carbohydrates required for interaction of the Fcγ2 region with effector cell Fe receptors and the C1q component of complement, and therefore disrupts Fc interactions. The in vitro CDC and ADCC activity of deglycosylated and unmodified alemtuzumab were compared using the CHO-CD52 cell line as a target. Deglycosylation of the antibody did not affect binding to CD52 cells as determined by flow cytometry (data not shown), but did abolish the ability of the antibody to lyse CD52⁺ cells through ADCC or CDC in vitro (FIGS. 10A and 10B).

For ADCC analysis, target cells were labeled with ⁵¹chromium (New England Nuclear, Boston, Mass.) overnight (100 μCi/1×10⁶ cells) and plated in v-bottom 96 well plates at 5×10³ cells/well. Peripheral blood mononuclear cells (PBMCs) or mouse neutrophils were added as effector cells (effector:target ratio of 50:1 for PBMCs and 200:1 for neutrophils) and various concentrations of antibody (0.5-10 μg/ml) were added in triplicate in a total volume of 200 μl. For CDC analysis, labeled target cells were plated with antibody (10 μg/ml) and 10% human complement (Quidel, San Diego, Calif.). Purified human IgG or infliximab (REMICADE®; Hanna Pharmaceutical, Wilmington, Del.) was used as an irrelevant negative control. After 5 hours of incubation for ADCC and 2 hours for CDC, plates were spun at 900 rpm and 100 μl of cell-free supernatant was collected from each well and counted in a MicroBeta Trilux Scintillation Counter (Wallac, Gaithersburg, Md.). The amount of ⁵¹Cr spontaneously released was obtained by incubating target cells alone in medium. Spontaneous release was typically below 20%. The total amount of ⁵¹Cr incorporated by the target cells was determined by adding 1% triton X-100 in distilled water, and the percentage lysis was calculated as follows:

[(sample cpm−spontaneous cpm)/(total cpm−spontaneous cpm)]×100.

### B. Deglycosylation of Alemtuzumab Abolishes its Anti-tumor Activity In Vivo

Comparison of unmodified and deglycosylated alemtuzumab in vivo in the CHO-CD52 s.c. tumor model indicated that, while treatment with the unmodified antibody significantly increased survival, the deglycosylated form did not exhibit any significant anti-tumor activity (FIG. 10C), indicating that Fc interactions are required for the anti-tumor activity of alemtuzumab in vivo.

### C. Removal of Complement and Mediators of ADCC Abolishes the Anti-tumor Activity of Alemtuzumab

Mice, treated with unmodified alemtuzumab, were depleted of complement with cobra venom factor and of effector NK cells and neutrophils with anti-asialo-GM-1 and anti-Gr-1, respectively. Our results suggest that unmodified alemtuzumab with an intact Fc portion and ability to bind CD52 became unable to inhibit tumor growth, when Fc-interacting components, i.e., complement and effector cells, were inactivated (FIG. 10D). The anti-tumor activity of alemtuzumab in vivo was primarily mediated by Fc interactions and binding of the antibody to tumor cells alone was not sufficient to significantly inhibit tumor growth.

### Example 13

### The anti-tumor activity of alemtuzumab requires the presence of nentrophils and NK cells

In order to define more precisely the Fc interactions responsible for the anti-tumor activity of alemtuzumab in vivo, we inactivated effector mechanisms individually prior to treatment with the antibody using methods described in Example 7, supra.

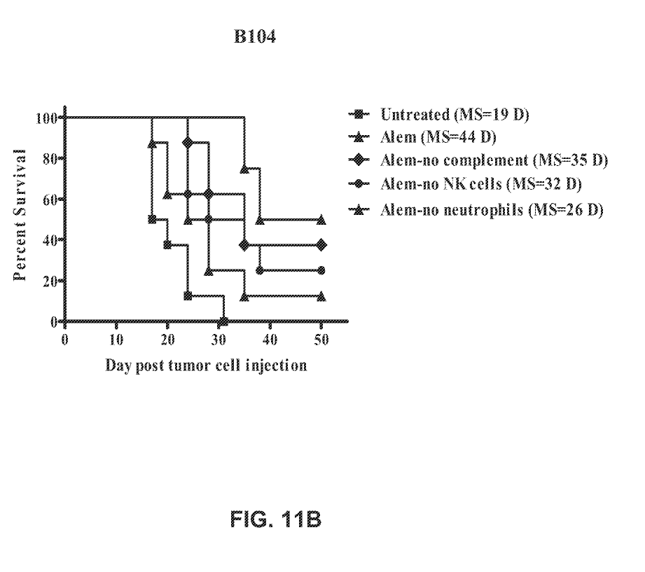

In the s.c. CHO-CD52 model (FIG. 11A), inactivation of complement alone with cobra venom factor had no detectable impact on the anti-tumor activity of alemtuzumab. By comparison, selective removal of NK cells with anti-asialo-GM-1 reduced anti-tumor activity but a significant degree of protection was still achieved (75% vs 100% survival, p=0.0093 vs untreated). Interestingly, removal of neutrophils with anti-Gr-1 antibodies completely abolished the anti-tumor activity of alemtuzumab (p=0.8620 vs untreated) indicating a primary role for these effector cells. Similar results were obtained with the B104 cell line which endogenously expresses CD52 (FIG. 11B). Individual removal of complement or NK cells reduced, but did not abolish, the anti-tumor activity of alemtuzumab (38% and 25% vs 50% survival, p=0.0010 and p=0.0045 vs untreated, respectively) while elimination of neutrophils removed the benefit of treatment with the antibody (p=0.1022 vs untreated). Results from these tumor models suggest a predominant role for ADCC as opposed to CDC in tumor growth inhibition by alemtuzumab and identify neutrophils as major mediators of the antibody's activity.

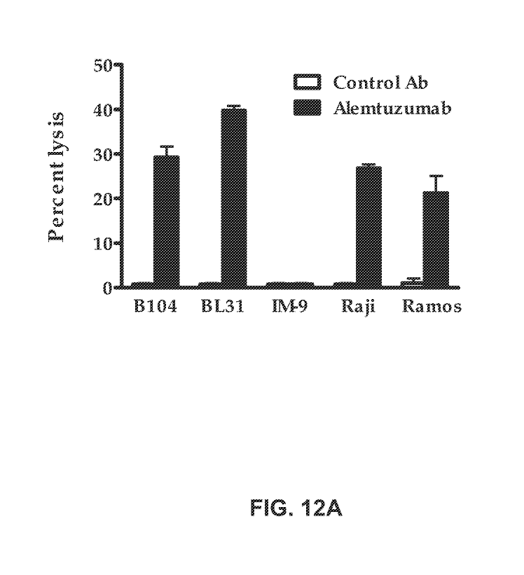

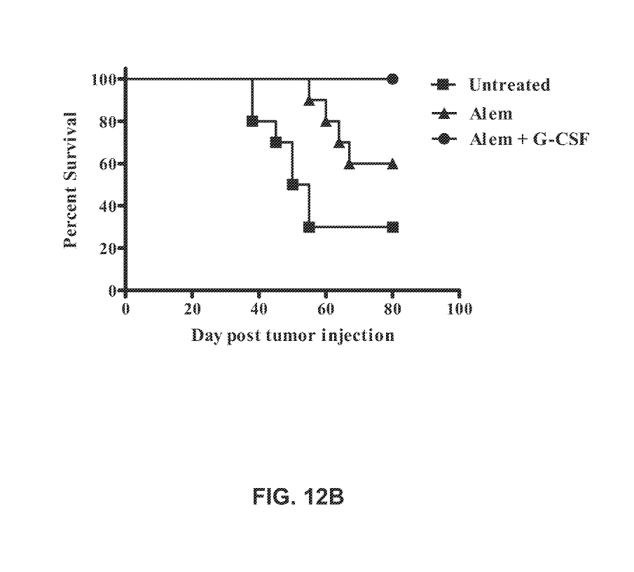

The ability of neutrophils to participate in alemtuzumab-mediated killing of tumor cells was also directly confirmed in vitro. Murine neutrophils isolated from the peritoneal cavity of mice injected with thioglycolate were added to various tumor cell lines in conjunction with alemtuzumab or infliximab (anti-INF-α as an irrelevant control antibody) and the level lysis was measured 5 hours later. The neutrophils mediated robust ADCC killing of CD52⁺ tumor cells (B104, BL-31, Raji and Ramos in the presence of alemtuzumab but not infliximab (FIG. 12A). There was no significant killing of IM-9 tumor cells which express very low levels of CD52 (7,000 molecules/cell), in line with the lack of efficacy of the antibody against this tumor line in vivo (FIG. 8D).

### Example 14

### Increasing the Number of Circulating Neutrophils Enhances the Anti-tumor Activity of Alemtuzumab

Our results suggest an essential role of neutrophils in the anti-tumor activity of alemtuzumab in vivo. We further investigated whether increasing the number of circulating effector neutrophils through the administration of G-CSF would enhance the therapeutic potential of alemtuzumab.

To increase the number of circulating neutrophils, mice were treated i.p. with 20 μg/mouse recombinant human G-CSF (NEUPOGEN®; Hanna Pharmaceutical, Wilmington, Del.) twice a week starting on day 4 post tumor cell injection and continued twice weekly for the duration of the study. This treatment resulted in an approximately 50% increase in circulating neutrophils as determined by flow cytometry staining for Gr-1 (data not shown). in the Raji s.c. model (FIG. 12B), concomitant administration of G-CSF and alemtuzumab starting 4 days after the injection tumor cells was found to significantly enhance the anti-tumor efficacy of alemtuzumab compared to treatment with the antibody alone resulting in 100% vs 60% survival of the animals by day 80 (p=0.0291). These results suggest that enhancing the number of available effector neutrophils enhances the therapeutic efficacy of alemtuzumab.

### Example 15

### Impact of Co-administration of Alemtuzumab and G-CSF on Lymphocyte Depletion In Vivo

As described above, we have discovered in xenograft tumor models that the lymphocyte depleting activity of alemtuzumab is mediated by a combination of NK cells and neutrophils. In this example, we further evaluated the lymphocyte depleting activity of alemtuzumab (CAMPAT®-1H, Genzyme Corporation, Cambridge, Mass., also referred as “Campath®”) in the presence or absence of G-CSF (NEUPOGEN®, Hanna Pharmaceutical, Wilmington, Del.) using the hCD52 transgenic mouse model. We have observed that treatment with NEUPOGEN® mobilizes neutrophils into circulation in hCD52 transgenic mice up to 24 hours post injection.

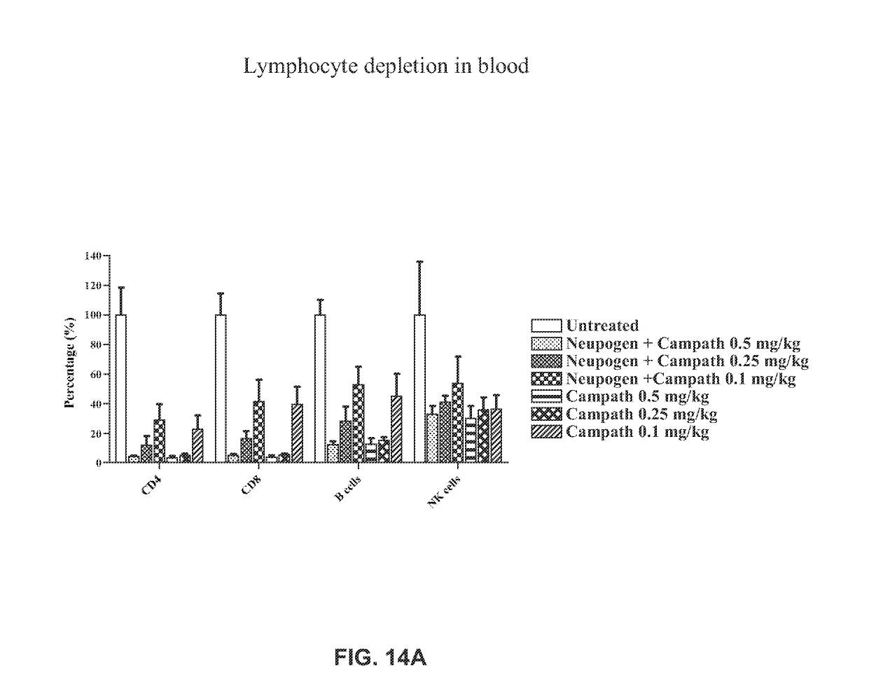

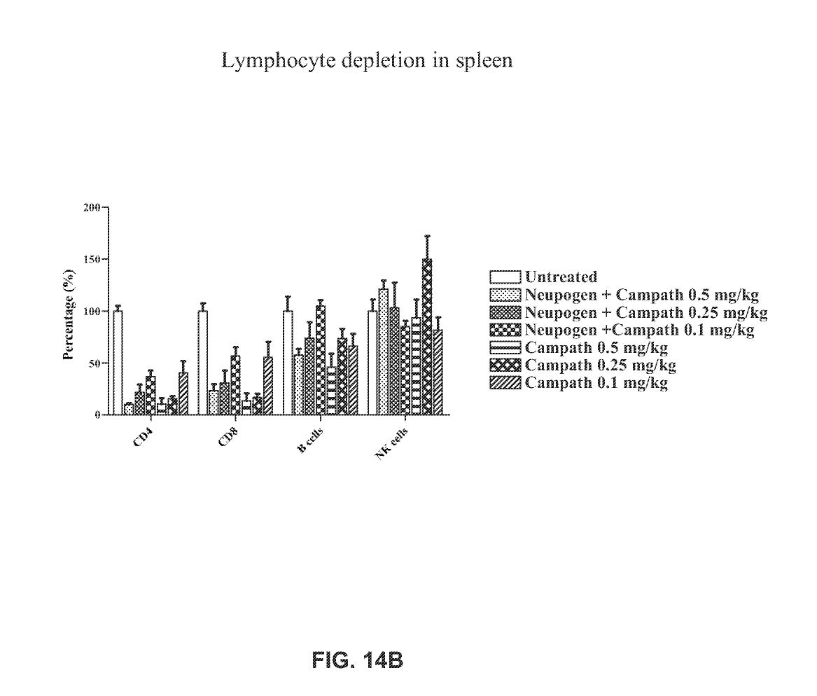

Mice were injected with NEUPOGEN® at 20 μg per mouse iv, Twenty-four hours later, mice received a dose of Campath® administered iv at 0.1, 0.25, or 0.5 mg/kg. Three days post Campath® administration, blood and spleens were collected to determine the level of lymphocyte depletion using flow cytometry analysis. Mice treated with Campath® alone displayed dose-dependent depletion of lymphocytes in both the blood and spleen (FIGS. 14A-14B). The addition of NEUPOGEN® to increase the number of circulating neutrophils did not seem to enhance the depleting activity in this timeframe (FIGS. 14A-14B). These data suggest that in this particular mouse model, given the number of target cells available and the concentrations of Campath® tested in this example, increasing the number of effector cells with NEUPOGEN® at the tested concentration did not enhance the lymphocyte depleting activity of Campath®.

### Example 16

### Impact of Co-administration of Alemtuzumab/CAMPATH®-1H and GM-CSF/LEUKINE® on Lymphocyte Depletion In Vivo

In this example, we evaluated the lymphocyte depleting activity of alemtuzumab (CAMPATH®-1H, Genzyme Corp., Cambridge, Mass., also referred as “Campath®” herein) in the presence or absence of GM-CSF (Leukine® or sargramostim, Genzyme Corp., Cambridge, Mass.) using the hCD52 transgenic mouse model. We have observed that treatment with Leukine® mobilizes macrophages and to a lesser extent neutrophils into circulation in hCD52 transgenic mice up to 2 hours post injection.

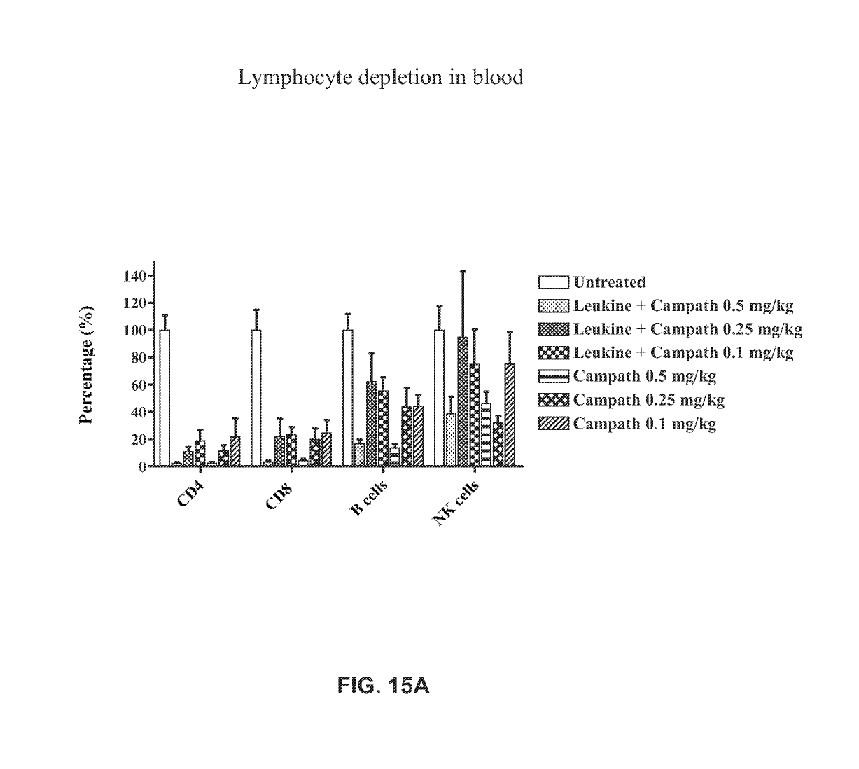

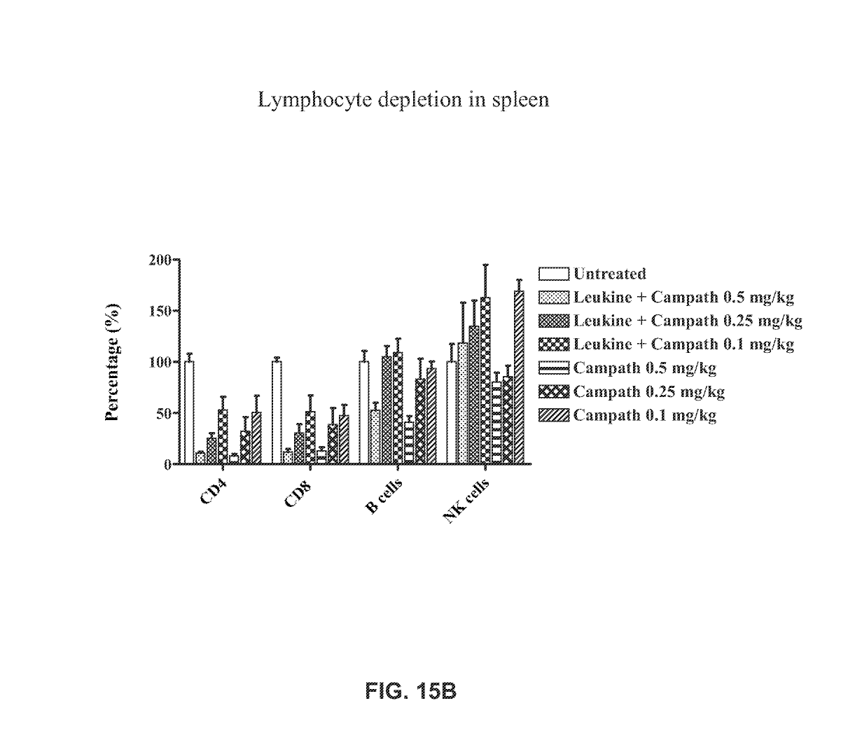

Mice were injected with Leukine® at 20 μg per mouse iv. Two hours later, mice received a dose of Campath® administered iv at 0.1, 0.25, or 0.5 mg/kg. Three days post Campath® administration, blood and spleens were collected to determine the level of lymphocyte depletion using flow cytometry analysis. Mice treated with Campath® alone displayed dose-dependent depletion of lymphocytes in both the blood and spleen (FIGS. 15A-15B). The addition of Leukine® to increase the number of circulating neutrophils did not seem to enhance the depleting activity in this timeframe (FIGS. 15A-15B). These data suggest that in this particular model, given the number of target cells available and the concentrations of Campath® tested in this example, increasing the number of effector cells with Leukine® at the tested concentration did not enhance the lymphocyte depleting activity of Campath®.

### Example 17

### Evaluating the Kinetics of Depletion in Response to Co-administration of Alemtuzumab and C-CSF

In this example, we investigated if increasing the number of neutrophils in circulation may increase the depleting activity or the kinetics of Campath®-mediated lymphocyte depletion in the hCD52 transgenic mouse model.

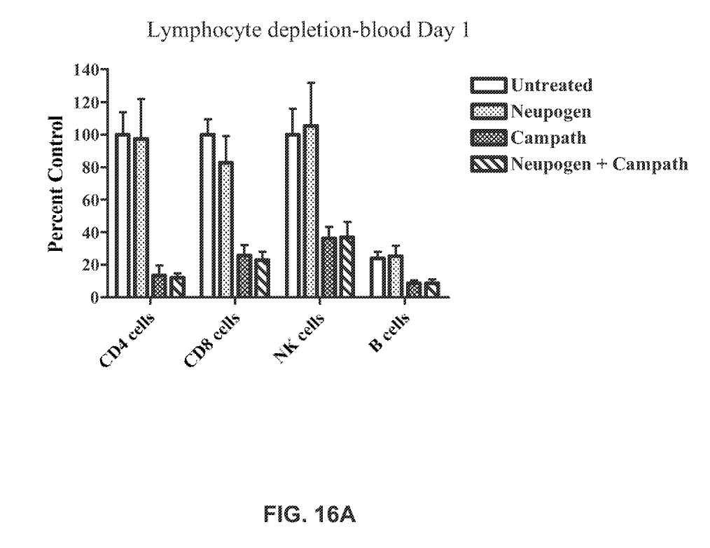

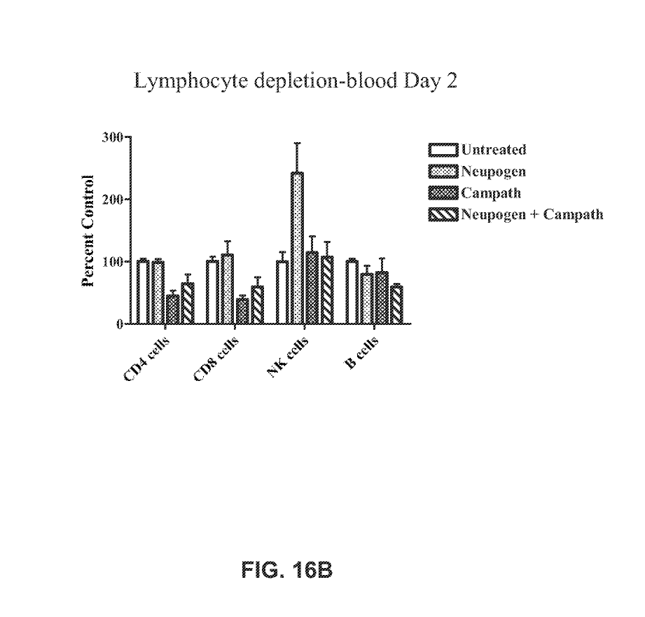

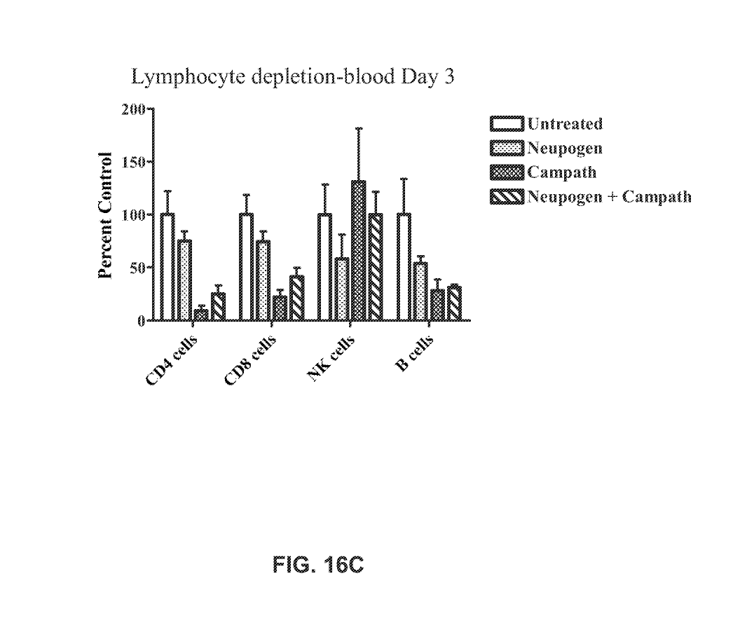

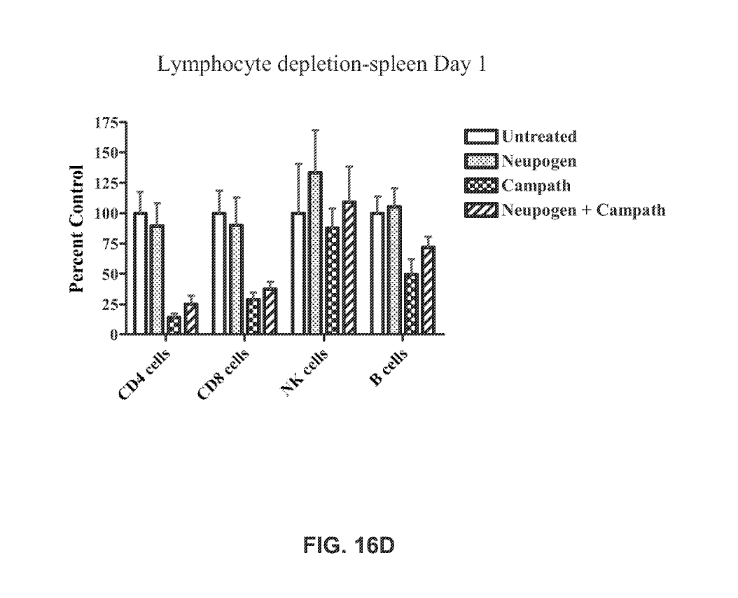

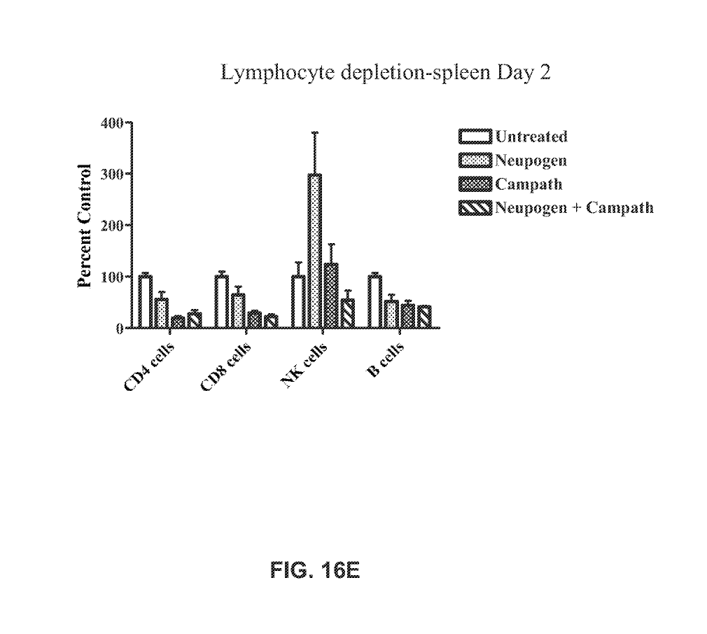

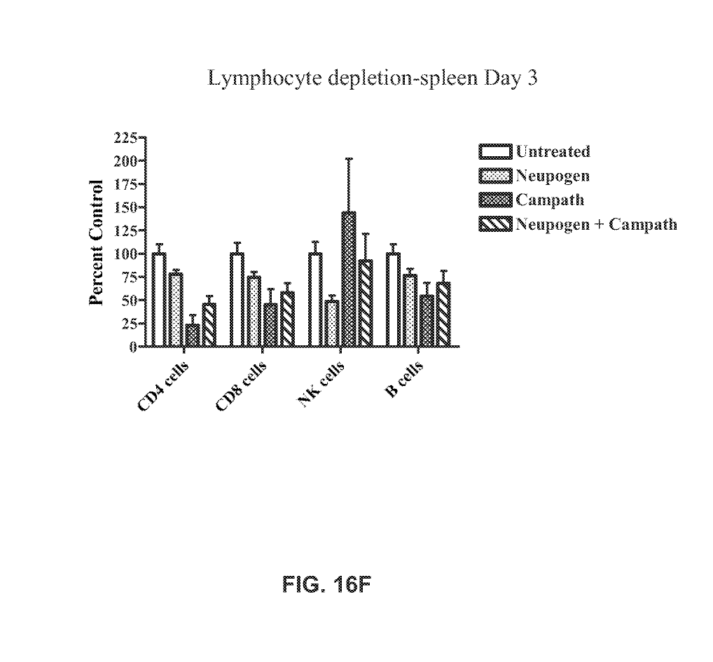

Mice were injected with NEUPOGEN® at 20 ug per mouse iv. Twenty-four hours later, mice received a dose of Campath® administered iv at 0.1 mg/kg. At one, two, and three days post Campath® administration, blood and spleens were collected to determine the level of lymphocyte depletion using flow cytometry analysis. Mice treated with Campath® alone displayed a significant level of lymphocyte depletion in both the blood and spleen at all time points examined (FIGS. 16A-16F). The addition of NEUPOGEN® to increase the number of circulating neutrophils did not seem to enhance the depleting activity at any of the time points (FIGS. 16A-16F). This data suggests that in this particular model, given the number of target cells available and the concentrations of Campath® tested, increasing the number of effector cells by NEUPOGEN® at the tested concentration did not enhance the lymphocyte depleting activity of Campath®.

### Example 18

### Analysis of the Efficacy of a Monoclonal Mouse Anti-mouse CD52 Antibody in Combination with G-CSF or GM-CSF in MRL/Ipr Female Mice

In this example, we investigate the lymphocyte depleting activity of a monoclonal IgG2a mouse anti-mouse CD52 antibody in the presence of G-CSF or GM-CSF in a MRL/lpr mouse model. The monoclonal IgG2a mouse anti-mouse CD52 antibody was generated in-house. The MRL/lpr mouse strain (Jackson Labs) harbors a mutation in the FAS gene and thus results in a lymphoproliferative condition. Lymphocytes fail to die through the normal apoptotic pathways and consequently accumulate in the circulation and lymphoid tissues as the mice age. This particular condition is analogous to chronic lymphocytic leukemia where large numbers of CD52-positive lymphocytes can be found in circulation. The monoclonal anti-mouse CD52 antibody used in this example was generated in house and is capable of mediating depletion of both T cells and B cells. See, e.g., International Application PCT/US2010/034704. Under these circumstances, increasing the number of effector cells (i.e., neutrophils and/or macrophages) in the circulation of mice through the administration of G-CSF (e.g., NEUPOGEN®)or GM-CSF (e.g., Leukine® may increase lymphocyte depletion mediated by the monoclonal anti-mouse CD52 antibody. Groups of 15 mice receive daily injections of G-CSF or GM-CSF on days 1 through 4 in combination with the monoclonal anti-mouse CD52 antibody at 10 mg/kg on days 2 through 4. Separate groups of 15 mice receive either vehicle or the anti-mouse CD52 antibody alone at 10 mg/kg on days 2 through 4, Readouts for the experiment include lymphocyte depletion in the blood and spleen measured by flow cytometry on days 5 through 7 (N=5 mice per day).

1. A method of treating a patient in need thereof, comprising:
administering to the patient an agent that stimulates neutrophils, or natural killer (NK) cells, or both; and
administering to the patient a therapeutically effective amount of an anti-CD52 antibody.

2. A method of increasing the efficacy of treatment with an anti-CD52 antibody, comprising administering to a patient who receives said treatment an agent that stimulates neutrophils, or natural killer (NK) cells, or both.

3. A method of reducing a side effect in a patient who receives treatment with an anti-CD52 antibody, comprising administering to the patient an agent that stimulates neutrophils, or NK cells, or both, thereby reducing the effective amount of the anti-CD52 antibody needed in said treatment.

4. The method of claim 3, wherein the side effect is infusion reaction.

5. The method of claim 3, wherein the side effect is secondary autoimmunity.

6. The method of claim 3, wherein the side effect is induction of an antibody response against the anti-CD52 antibody.

7. A method of increasing lymphocyte depletion in a patient who receives treatment with an anti-CD52 antibody, comprising administering to the patient an agent that stimulates neutrophils, or NK cells, or both.

8. A method of treating a patient in need thereof, wherein the patient receives treatment with an anti-CD52 antibody, and wherein the patient has an abnormally low neutrophil count, comprising administering to the patient an agent that stimulates neutrophils, or NK cells, or both.

9. A method of treating a patient in need thereof, comprising:
administering to the patient an agent that stimulates CD4+CD25+FoxP3+ regulatory T cells; and
administering to the patient a therapeutically effective amount of an anti-CD52 antibody.

10. A method of increasing CD4+CD25+FoxP3+ regulatory T cells in a patient who receives treatment with an anti-CD52 antibody, comprising administering to the patient an agent that stimulates said regulatory T cells.

11. A method of increasing e efficacy of treatment with an a CD52 antibody, comprising administering to a patient who receives said treatment an agent that stimulates CD4+-CD25+FoxP3+ regulatory T cells.

12-14. (canceled)

15. The method of claim 1, wherein the agent that stimulates neutrophils, or NK cells, or both is granulocyte monocyte colony stimulating factor (GM-CSF), granulocyte colony stimulating factor (G-CSF), interferon-gamma (IFN-γ), a CXC chemokine receptor 4 (CXCR4) antagonist, or a CXC chemokine receptor 2 (CXCR2) agonist.

16. The method of claim 1, wherein the agent that stimulates neutrophils, or NK cells, or both is sargramostim, plerixafor, interferon gamma-1b.

17. The method of claim 1, wherein the anti-CD52 antibody is alemtuzumab.

18-19. (canceled)

20. The method of claim 1, wherein the patient suffers from an inflammatory condition.

21-23. (canceled)

24. The method of claim 1, wherein the patient has cancer.

25-29. (canceled)

30. A kit for treating a patient in need thereof, comprising (a) an anti-CD52 antibody; and (b) an agent that stimulates neutrophils and/or NK cells.

31-33. (canceled)

34. An immunoconjugate comprising an anti-CD52 antibody fused to an agent that stimulates neutrophils or NK cells, or both.

35-37. (canceled)

38. A kit for treating a patient in need thereof, comprising (a) an anti-CD52 antibody; and (b) an agent that stimulates CD4+CD8+FoxP3+ regulatory T cells.

39-41. (canceled)

42. An immunoconjugate comprising an anti-CD52 antibody fused to an agent that stimulates CD4+CD8+FoxP3+ regulatory I cells.

43-45. (canceled)


SEQUENCE LISTING (4 sequences)

SEQ ID NO: 1 - protein, 61 aa, Homo sapiens
MKRFLFLLLTISLLVMVQIQTGLSGQNDTSQTSSPSASSNISGGIFLFFVANAIIHLFCFS

SEQ ID NO: 2 - DNA, 2604 bp, Homo sapiens
ctcctggttcaaaagcagctaaaccaaaagaagcctccagacagccctgagatcacctaaaaagctgctaccaagacagccacgaagatcctaccaaaatgaagcgcttcctcttcctcctactcaccatcagcctcctggttatggtacaggtaagagcaacgcctggcaccactgccaggactccccaaagttgcttggcatggagggagggcatacaggatgtgagctcccagagacccagccttctctcctgagagcacagcagatggactccaacaggaaaactggggacacagcgggcctggccgcccagccttccagctcttctggctggggctgcactgcttgcgggctgccctgagagaaacctgtatccacagctggggagcacttacagggtgctaggcgcgttggggcacccaaggggtgttgtggaataatagagaactaagagacttgggaagacaaccctgagtgaggaaggcatcggcccacagggagtctggggagatggtcctttggggagaggccaggagatgtccaagtggagcctgctgtggttatggaggtgacaggggacactctaacccccactcccctgccagagagaagcctggatgttcaggaatgcaaagaagacaggactctcccatttcagaaacaactggagtctcaaaagcgactgctattccaggaggcagctttggtagcatcccaggtgcccagaggggagtcagtacagcagaccggagccatcaagttcttgatcaagaacagcttgatcaccatcaaaaccagcttaatctccatcaagaacagttctagttgcagctccatcaagcattaaggacagctcgatcaccatctggccacttccccaccccacatccagccacccacgctttgccaagtgctttctcactcagccctggctcatgtgaccttcacaactgtgatgaacttctccccactcagaagatgaaactgaggctcagggaagctaagccctttgccaggctaggaaggtcaagggccagggctcccctgtctccctctatgccagaagggctggtcgctggggaaaggtgaaaagaaaggttctgtgaccgggcacggtggctcacgcctataatcccagcactttgggaggccgaggcaggcggatcacctgaggtcaggagttcaagaccagcctggccaacatgacaaaatcccgtctctactaaaaatacaaaaattagctgggcgagccgggcgcgctggctcgcgtctgtaatcccagcactttgggaggctgaggcgggtggatcatgaggtcaggagatcgagaccatcctggctaacacattgaaacctcgtctctaccataaatacaaaaagaaattagccgggcgtggcgtggtggcgggcgcctgtagtcccagctactcgggaggctgaggcaggagaatggcgtgaacctgggggtgaagcttgcagtgagccgagatcacgccactgcactccagcctgggcgggcgacagagcaagactctgtctcggaaaaaaaaaaaaaattagctgggcgtgatagcggctgcctataatcccagctactcgggaggttgaggggtggaggttgcagtgacccgagatcatgccacttcactccagcctgggcaaaagagcgaaactctatctcaaaaaaacagaaagtttctgtgaaagggggtggacaggggcagtgatcacctctgtccctgcctgtgtcaaagcaggaagcagtcaaacatcctgacaggaccttgtggaagaagtagtggtcacatggggcagaactgaggactgttttatggaggtgggcggatggagggcggggagggtagaaaggggactgcatctttaaataacaaacacgctattcaaaagtcccctgagggctgggtacgatggctcacacctgtaatcccagtactttgggaggccgaggcgggtggatcaccaggtcaggagttcgagaccagcctgaccaacatggtgaaaccccgtctctactaaaaatagaaaaattagccgggcgtggtggcacgtgcctatagtcccagctactcaggaggctgaggcaggagaatcacttgaacccgggaggtggaggatgcagtgagccgagatcgcatcactgcagtccagcctgggtgacagagtgagactccacctctaaaaaaaaaaaaaaaagtcccctgatcttatcccacttctcctcctacagatacaaactggactctcaggacaaaacgacaccagccaaaccagcagcccctcagcatccagcaacataagcggaggcattttccttttcttcgtggccaatgccataatccacctcttctgcttcagttgaggtgacacgtctcagccttagccctgtgccccctgaaacagctgccaccatcactcgcaagagaatcccctccatctttgggaggggttgatgccagacatcaccaggttgtagaagttgacaggcagtgccatgggggcaacagccaaaataggggggtaatgatgtaggggccaagcagtgcccagctgggggtcaataaagttacccttgtacttgca

SEQ ID NO: 3 - DNA, 186 bp, Homo sapiens
atgaagcgcttcctcttcctcctactcaccatcagcctcctggttatggtacagatacaaactggactctcaggacaaaacgacaccagccaaaccagcagcccctcagcatccagcaacataagcggaggcattttccttttcttcgtggccaatgccataatccacctcttctgcttcagttga

SEQ ID NO: 4 - protein, 12 aa, Homo sapiens
GQNDTSQTSSPS